(19) United States
(12) Patent Application Publication
(10) Pub. No.: US 2024/0350794 A1
(43) Pub. Date: Oct. 24, 2024

(54) CATHETER BEARING ARRANGEMENT

(71) Applicant: White Swell Medical Ltd, Kibbutz Shefayim (IL)

(72) Inventors: Ronan Keating, Galway (IE); Sagi Raz, Tel-Aviv (IL); Diarmuid Conroy, Galway (IE); Reed Williston, Galway (IE); Kevin Bancud, Galway (IE); Adrian McMahon, Galway (IE); Eamon Brady, Galway (IE)

(21) Appl. No.: 18/629,264

(22) Filed: Apr. 8, 2024

Related U.S. Application Data
(60) Provisional application No. 63/459,388, filed on Apr. 14, 2023.

(51) Int. Cl. A61M 60/825; A61M 25/10; A61M 60/13; A61M 60/226; A61M 60/824; F04D 29/056

(52) U.S. Cl. CPC A61M 60/825; A61M 60/13; A61M 60/226; A61M 60/824; F04D 29/056; A61M 2025/1052

(57) ABSTRACT

The present invention generally relates to the field of fluid management procedures for the treatment of fluid management disorders in a patient, and related components and methods. In particular, the present invention is directed to bearing arrangements used in intravascular blood pump assembly designed and configured to generate low bearing interfacial resistive force when operated in a blood or plasma protein containing environment.

### FIELD OF THE INVENTION

The present invention generally relates to the field of fluid management procedures for the treatment of fluid management disorders in a patient, and related components and methods. In particular, the present invention is directed to bearing arrangements used in intravascular blood pump assembly designed and configured to generate low bearing interfacial resistive force when operated in a blood or plasma protein containing environment.

### BACKGROUND OF THE INVENTION

Acute decompensated heart failure and other fluid management disorders exert an enormous burden on health care systems consuming significant hospital resources and budget. In the US alone there are over 1 million hospitalizations annually for acute decompensated heart failure, with most of these patients being hospitalized for multiple days while their fluid management systems are being normalized. Implantable or indwelling blood pumps are very technically advanced systems and play a critical part in helping doctors restore the fluid management systems of the patient. Blood pumps can be used to support circulatory function or to support the process of decongesting the patient.

All blood pumps comprise a pumping element that rotates typically at high speed in a housing to effect forward momentum of blood. The pumping arrangement needs to be manufactured with great precision so as to ensure that the pumping element rotates in the housing without contacting the surfaces of the housing, while simultaneously maintaining precision gaps between the pumping element and the housing. The pumping element also needs to operate precisely on its central axis without vibrating or deviating therefrom. To do this it is desirable that the pumping element be mounted on a precision bearing arrangement during operation. The inventors have evaluated a variety of conventional bearing arrangements which have proven deficient in blood-contacting applications where miniaturization is desired, for example in a catheter-based blood pumping arrangement.

### SUMMARY OF THE INVENTION

The present invention provides an intravascular catheter system and related components designed for treating patients suffering with bodily fluid disorders including acute decompensated heart failure (ADHF), congestive heart failure (CHF), edema and ascites.

The invention recognizes the problems with conventional bearing arrangements used in intravascular blood pumps and solves these problems with newly discovered principles on how to optimize bearing arrangements for blood pumping devices. In particular, the present invention is directed to bearing arrangements used in an intravascular blood pump assembly designed and configured to generate low bearing interfacial resistive force when operated in a blood or plasma protein containing environment.

Various aspects of the present invention are directed to bearing arrangements used in intravascular blood pump assembly designed and configured to generate low bearing interfacial resistive force during the fluid management procedure.

In one aspect, the present invention provides a blood pump assembly comprising a pump housing, a pumping element positioned within and rotatable relative to the pump housing, and a bearing arrangement configured to hold the pumping element and geometrically designed to generate a low geometric drag factor.

In a second aspect, the present invention provides a bearing arrangement used in a blood pump assembly comprising a plurality of bearing surfaces and at least one of the bearing surfaces configured geometrically to generate a low geometric drag factor.

In a third aspect, the present invention provides a bearing arrangement comprising a plurality of bearing surfaces and geometric drag factor. The geometric arrangement of the plurality of bearing surfaces is configured to generate a low cumulative geometric drag factor.

These and other aspects and advantages of the present invention are described in the following detailed description of the invention.

In one aspect, the invention discloses an intravascular blood pump assembly comprising an elongated tubular housing having a proximal end, a distal end, at least one inlet 207 and at least one outlet, a pumping element 250 positioned within the elongated tubular housing and configured to rotate relative to the elongated tubular housing, and a bearing arrangement configured to the hold the pumping element and configured geometrically to generate a low bearing interfacial resistive force.

In some embodiments, the pumping element comprises at least one bearing surface. For example, in some embodiments, the bearing surface comprises a geometric drag factor. In some embodiments, the bearing surface is configured geometrically to generate a low geometric drag factor. The bearing surface comprises, in some embodiments, a first bearing surface and a second bearing surface. In some embodiments, the first bearing surface and the second bearing surface comprise a continuous surface. In some embodiments, the bearing surface comprises a static surface and a rotating surface. In some embodiments, the static surface and the rotating surface are substantially equal.

In some embodiments of the intravascular blood pump assemblies of the invention, the bearing arrangement comprises at least one bearing gap. The bearing gap comprises a small space between the static surface and the rotating surface, in some embodiments.

In some embodiments, the geometric drag factor is in a range from 0.10 mm⁴ to 2.97 mm⁴. For example, in some embodiments, the geometric drag factor is less than 2.97 mm⁴. In some embodiments, the geometric drag factor is less than 1.45 mm⁴. In some embodiments, the geometric drag factor is less than 0.75 mm⁴. In some embodiments, the geometric drag factor is less than 0.42 mm⁴. In some embodiments, the geometric drag factor is less than 0.18 mm⁴.

In particular embodiments, the low bearing interfacial resistive force results partially from a low geometric drag factor.

In other embodiments, the pumping element comprises an impeller and the impeller comprises a pair of radially opposed impeller blades. For example, in some embodiments, the impeller blades comprises an impeller blade proximal end, an impeller blade distal end, and a blade angel with respect to the axis of the impeller. In some embodiments, the blade angle varies along the length of the impeller blades. In various embodiments, the blade angle of the impeller blade proximal end is configured to accelerate blood radially.

In some embodiments, the impeller comprises an impeller core.

In some embodiments, the intra vascular blood pump comprises a drive shaft and a motor. In particular embodiments, the drive shaft extends between the motor and the impeller. Further, in various embodiments, the motor comprises an electric motor configured to rotate at high speed. In some embodiments, the drive shaft extends into the impeller core along the impeller neutral axis.

In some embodiments, the intravascular blood pump assembly further comprises a bearing shaft positioned distally of the impeller.

In particular embodiments, the bearing arrangement comprises a bearing block. The bearing block comprises a plurality of bearing surfaces, in some embodiments. Further, in some embodiments, the bearing block comprises a first distal bearing surface configured to restrain the bearing shaft substantially fixed relative to the neutral axis of the impeller. The bearing block may comprise a second distal bearing surface configured to restrain the impeller at least partially in a substantially fixed axial position relative to the bearing block. In some embodiments, the bearing block comprises a third distal bearing surface configured to restrain the impeller at least partially in a substantially fixed axial position relative to the bearing block.

In some embodiments, the intravascular blood pump assembly is configured for intravascular delivery to a treatment location.

In some embodiments, the intravascular blood pump assembly further comprises a catheter shaft. For example, the catheter shaft may extend exterior of the patient and may be connected to the elongate tubular housing. In some embodiments, the catheter shaft comprises a plurality of lumens.

In particular embodiments, the elongate tubular housing comprises an expandable and collapsible restrictor encircling the elongate tubular housing. The expandable and collapsible restrictor comprises a compliant balloon, in some embodiments. The expandable and collapsible restrictor may be disposed between the inlet and outlet of the elongate tubular housing. In some embodiments, the expandable and collapsible restrictor is at least partially disposed over the impeller. Further, in some embodiments, the expandable and collapsible restrictor comprises a tapering funnel region on its proximal end. Thus, the tapering funnel region guides blood flow in the vessel to the inlet of the elongate tubular housing without fluid turbulence or recirculation, in some embodiments.

In various embodiments, the bearing arrangement comprises a proximal bearing surface proximal of the impeller. The proximal bearing surface is configured to restrain the drive shaft substantially fixed relative to the neutral axis of the impeller 202, in some embodiments. The proximal bearing surface comprises a plurality of bearing surface segments disposed equiangularly around the drive shaft, in some embodiments.

In another aspect, the invention discloses an intravascular blood pump assembly comprising an elongate tubular housing having a proximal end, a distal end, at least one inlet and at least one outlet, an impeller positioned within the elongate tubular housing and configured to rotate relative to the elongate tubular housing, and a bearing block distal of the impeller having a first bearing surface and a second bearing surface. The first bearing surface defines an axis of restraint, and the second bearing surface defines a plane of restraint, wherein the axis of restraint and the plane of restraint are orthogonal to each other.

In some embodiments, the axis of restraint is collinear with the central axis of the elongate tubular housing. For example, in particular embodiments, the plane of restraint comprises a discoid surface. The discoid surface of the plane of restraint may comprise a surface configured to abut an opposing surface. In some embodiments, the plane of restraint comprises an annulus. In other embodiments, the plane of restraint comprises a curved surface. Further, the plane of restraint may comprise a plane radial to the axis of the elongate tubular housing.

In some embodiments, the second bearing surface comprises a cylindrical surface.

In some embodiments, the distal end of the elongate tubular housing comprises a bearing reception space. The bearing reception space is configured to fixedly hold the central axis of the first bearing surface co-axial with the axis of restraint of the impeller, in some embodiments.

In various embodiments, the bearing block is mounted in circumferential interference in the distal region of the elongated tubular housing.

In particular embodiments, the first bearing surface and the second bearing surface comprise a low friction wear resistant material. For example, in some embodiments, the low friction wear resistant material is selected from the group comprising of polyether ether ketone, titanium, cobalt chrome, graphite, polyimide, or a combination thereof.

The intravascular blood pump assembly further comprises a first bearing gap at the first bearing surface, in some embodiments. The first bearing gap may comprise an annular gap. The first bearing gap has a thickness of less than 0.10 mm, in some embodiments.

In another aspect, the invention discloses an intravascular catheter blood pump comprising: a pump assembly, a catheter shaft, a drive shaft, and a motor. The pump assembly comprises a pump housing, an impeller, a restrictor, a pump inlet, and a pump outlet downstream of the pump inlet and the restrictor. The drive shaft coaxially connected to the impeller at its distal end and coaxially coupled to a rotor shaft of the motor at its proximal end and configured to transmit torque or rotational energy from the motor to the impeller. The catheter shaft comprises a central lumen configured to receive the drive shaft and facilitate the rotational operation of the drive shaft, in some embodiments. The central lumen is sized relative to the drive shaft to create an annular gap between the drive shaft and the central lumen, in some embodiments. The annular gap comprises, in some embodiments, an annulus of viscous grease surrounding the drive shaft. In example embodiments, the annulus of viscous grease extends along at least part of the length of the central lumen. In some embodiments, the annulus of viscous grease comprises an aspect ratio of defined by length/thickness. For example, the aspect ratio may be in the range of 5000 to greater than 30,000. In particular embodiments, the aspect ratio is greater than 5,000, greater than 10,000, greater than 15,000, greater than 20,000, greater than 25,000 and/or greater than 30,000.

In some embodiments, the annulus of viscous grease is bound in place by the inner surface of the drive shaft liner. The annulus of viscous grease may comprise a column of a viscous grease, in some embodiments. In particular embodiments, the viscous grease comprises a self-cohesive fluid when placed in the biological fluid environment. For example, the viscous grease may comprise a saline contact angle greater than 60°. The viscous grease may comprises a saline contact angle greater than 70°. The viscous grease may comprise a saline contact angle greater than 80°. The viscous grease may comprise a saline contact angle greater than 90°. In some embodiments, the annulus of viscous grease is configured to prevent the biological fluid ingress into the annulus between the drive shaft and the central lumen.

In some embodiments, the catheter shaft comprises a plurality of peripheral lumens. In some embodiments, the drive shaft comprises a longitudinal rod. In particular embodiments, the drive shaft is configured to be resistant to kinking when operating at rotational speeds of between 10,000 to 60,000 RPMs in tortuous body anatomy. In some embodiments, the drive shaft comprises a highly elastic metallic rod. In some embodiments, the drive shaft comprises a super elastic or shape memory metal. In various embodiments, the drive shaft is geometrically configured to operate within its elastic limits when the catheter shaft is placed in tortuous anatomy. In particular embodiments, the drive shaft has a second moment of area of less than 4×10⁻⁴ mm⁴. In some embodiments, the drive shaft has a second moment of area of less than 2×10⁻⁴ mm⁴. In some embodiments, the drive shaft has a second moment of area of less than 8×10⁻⁴ mm⁴.

### BRIEF DESCRIPTION OF THE DRAWINGS

FIGS. 1a-1h show an intravascular catheter blood pump generally in accordance with embodiments of the intravascular catheter blood pumps of the invention except with a conventional bearing arrangement, illustrating the problems with conventional bearing assemblies.

FIG. 2a is a schematic illustration of an intravascular catheter system in accordance with the present invention.

FIG. 2b is a close-up schematic illustration of a console and controller in accordance with an embodiment of the present invention.

FIG. 2c is a close-up schematic illustration of in-use configuration of a sheath and a blood pump in accordance with an embodiment of the present invention.

FIG. 2d is a cross-sectional view of the bearing arrangement of a catheter blood pump of the present invention.

FIG. 2e is a cross-sectional schematic illustration of a distal portion of a pump assembly in accordance with an embodiment of the present invention.

FIG. 3a is a schematic illustration of an impeller type intravascular pump in accordance with an embodiment of the present invention.

FIG. 3b is a schematic illustration of the axis of lateral restraint and the plane of longitudinal restraint of an impeller in accordance with an embodiment of the present invention.

FIG. 3c is a schematic illustration of the bearing surfaces in relationship to the impeller in accordance with an embodiment of the present invention.

FIG. 3d is a schematic illustration of the distal bearing surfaces used in calculation of a geometric drag factor for a bearing arrangement in accordance with the present invention.

FIG. 4a is a schematic illustration of a cylindrical bearing arrangement in accordance with an embodiment of the present invention.

FIG. 4b is an end view schematic illustration of an annular bearing surface in accordance with an embodiment of the present invention.

FIG. 4c is a cross sectional schematic illustration of a frustoconical bearing arrangement in accordance with an embodiment of the present invention.

FIG. 4d is a cross sectional view schematic illustration of a spherical bearing arrangement in accordance with an embodiment of the present invention.

FIG. 4e is an end view schematic illustration of the spherical bearing arrangement as described in FIG. 4d.

FIG. 5 is a cross sectional schematic illustration of a pump assembly having the impeller supported by proximal and distal ball nosed bearing arrangement in accordance with an embodiment of the present invention.

FIG. 6a is a cross sectional schematic illustration of a pump assembly having the impeller supported by proximal and distal cylinder bearing arrangement in accordance with an embodiment of the present invention.

FIG. 6b is a cross sectional schematic illustration of a distal bearing arrangement in accordance with an embodiment of the present invention.

FIG. 6c is a cross sectional schematic illustration of a proximal bearing arrangement in accordance with an embodiment of the present invention.

FIG. 7a is a schematic illustration of a pump having a novel drive shaft sealing arrangement in accordance with an embodiment of the present invention.

FIG. 7b is a cross-sectional view schematic illustration of the catheter shaft as described in FIG. 7a.

FIG. 8a is a schematic illustration of a bearing arrangement of the present invention mounted in a fixture assembly for testing said bearing arrangement in a blood flow test loop.

FIG. 8b shows a blood flow test loop configured to test a bearing arrangement.

FIG. 9a is a schematic illustration of a first bearing arrangement of the present invention mounted in a fixture assembly for testing said bearing arrangement in a blood flow test loop.

FIG. 9b shows a chart of RPMs and motor current versus time for the first bearing arrangement evaluated in a blood flow test loop.

FIG. 9c shows an image the first bearing arrangement after the testing in a blood flow test loop.

FIG. 10a is a schematic illustration of second bearing arrangement of the present invention mounted in a fixture assembly for testing the second bearing arrangement in a blood flow test loop.

FIG. 10b shows a chart of RPMs and motor current versus time for the second bearing arrangement evaluated in a blood flow test loop.

FIG. 10c shows the second bearing arrangement of the present invention still mounted in the fixture assembly after testing in a blood flow loop.

FIG. 10d shows the proximal end of the second bearing arrangement disassembled after testing in a blood flow test loop.

FIG. 10e shows the retainer ring of the second bearing arrangement disassembled after testing in a blood flow test loop and after disassembly.

### DETAILED DESCRIPTION OF THE PREFERRED EMBODIMENTS

The present invention provides an intravascular catheter system and related components designed for treating patients suffering with bodily fluid disorders including acute decompensated heart failure (ADHF), congestive heart failure (CHF), edema and ascites.

The invention recognizes the limitations of the current bearing arrangements for intravascular catheter systems and solves these limitations by providing novel bearing arrangements for intravascular blood pumps that allow micromotors to operate pumping elements thereof at high speeds and with low power consumption.

In particular, the present invention is directed to bearing arrangements used in an intravascular blood pump assembly designed and configured to generate low bearing interfacial resistive force when operated in a blood or plasma protein containing environment. Accordingly, the invention discloses newly discovered principles on how to optimize bearing arrangements for blood pumping devices.

### Conventional Bearing Arrangements

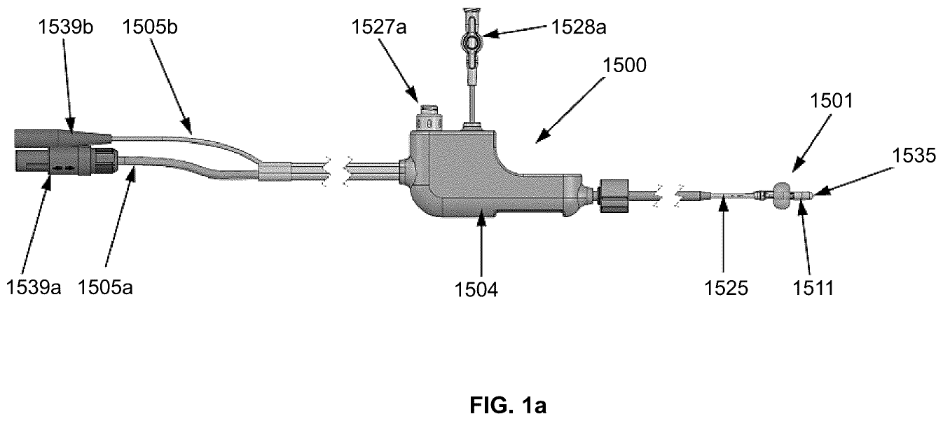

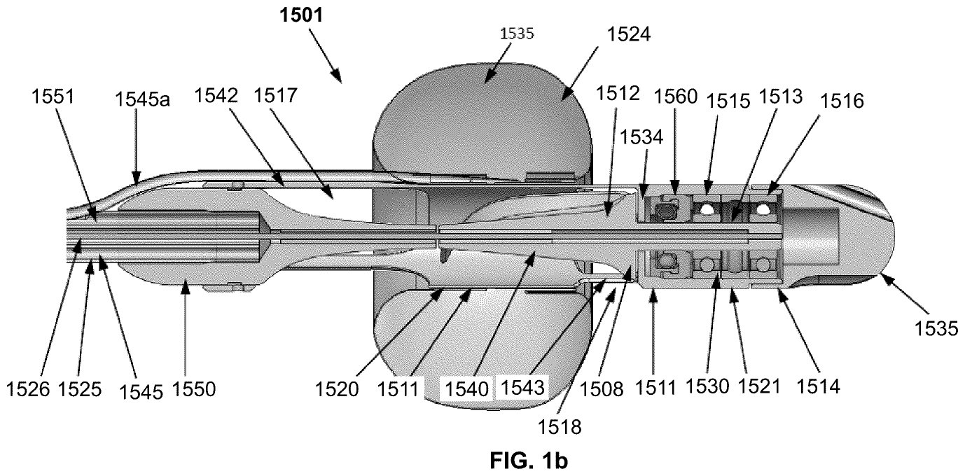

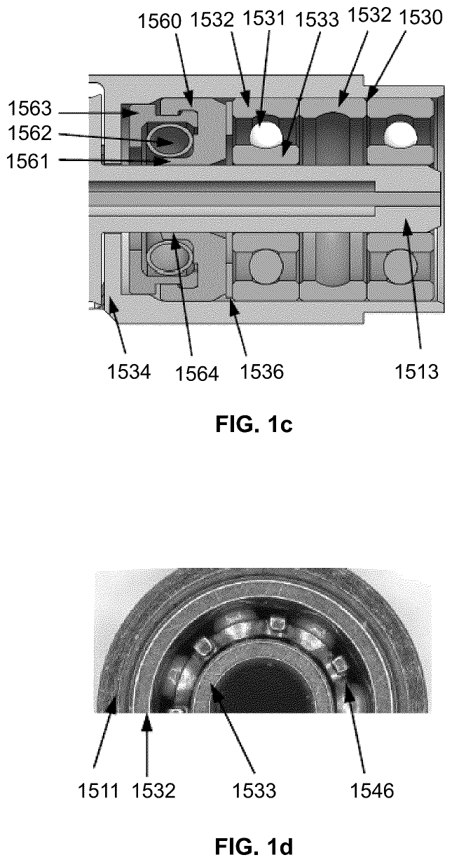

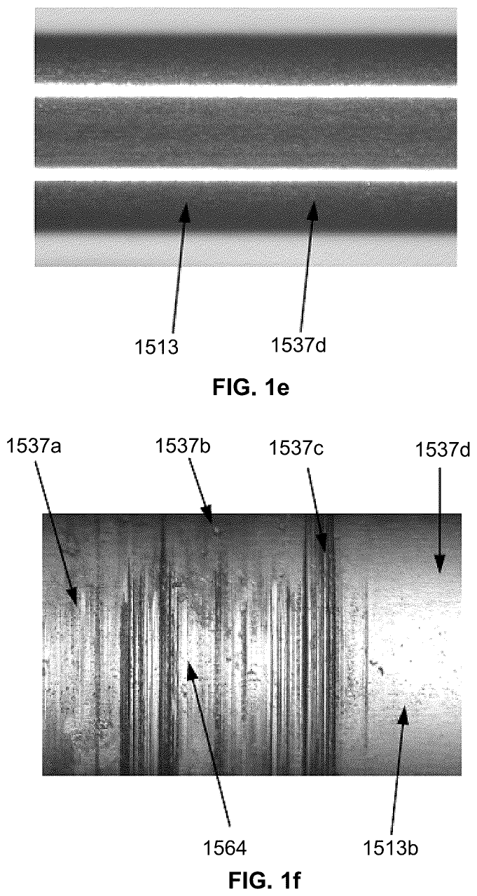

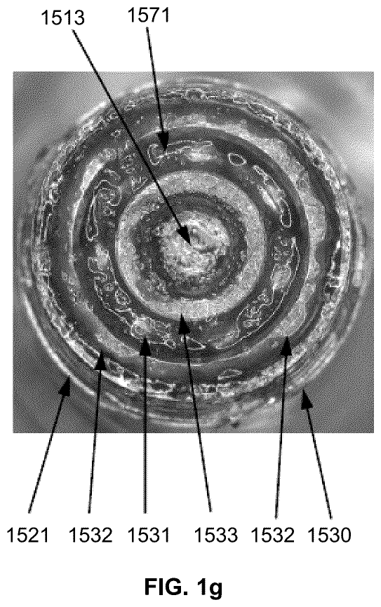

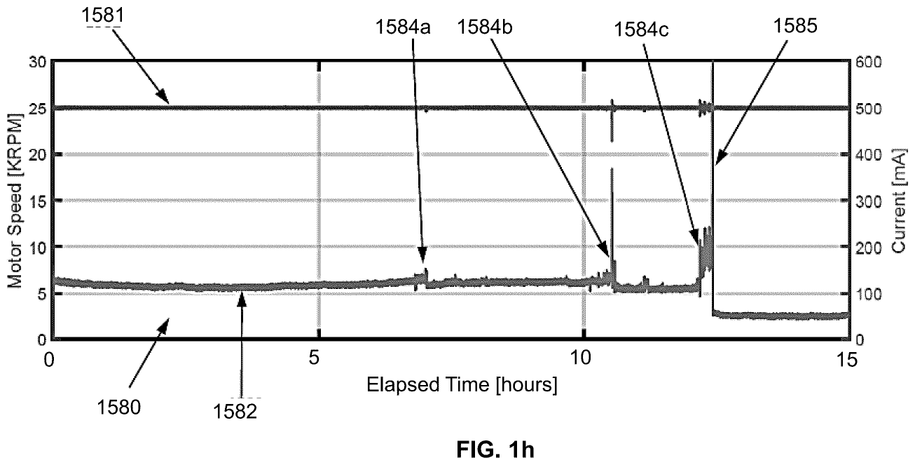

FIG. 1a shows an intravascular catheter blood pump 1500 which is generally in accordance with the intravascular catheter blood pumps of the invention except that the catheter blood pump 1500 comprises a conventional bearing arrangement 1530. The inventers attempted multiple configurations of the intravascular blood pump 1500 and failed to have the blood pump 1500 with conventional ball bearing arrangement 1530 operate successfully in mammalian blood for a long duration. This was because of bearing degradation in blood even though the bearings were highly durable in air, water or blood simulating fluid.

The intravascular catheter blood pumps 1500 comprise a pump assembly 1501, catheter shaft assembly 1502, a catheter shaft 1525, a drive shaft 1526, a catheter manifold 1504, connection cables 1505, connection cabling 1505a,b, connecters 1539a,b, and stopcock luer fittings 1527a and/or swabbable luer fittings 1528a. The intravascular catheter may include a catheter multi-lumen tubing 1551 and catheter lumens 1545. The pump assembly 1501 further comprises a restrictor, pump housing 1512, pumping element 1508, pump inlets 1517, pump outlets 1518, and a bearing arrangement 1530. The pump assembly may comprise one or more inlet struts 1542 and one or more outlet struts 1543. As discussed in detail below, the intravascular catheter blood pump may comprise an expandable and collapsible restrictor 1524 comprising a compliant balloon. The pump assembly may comprise an impeller comprising an impeller core 1540. The pump assembly may comprise a proximal housing 1520.

The blood pump may comprise a connecting cuff 1550, which establishes an interconnection between the pump assembly 1501 and the catheter shaft 1525. The connecting cuff 1550 may comprise a proximal opening into which the distal end of the catheter shaft 1525 is joined.

The bearing arrangement 1530 comprises a bearing shaft 1531, fixedly connected to the impeller 1511, a first bearing 1515, a second bearing 1516, a housing separator 1534, and spacer 1536. The first bearing 1515 and second bearings 1516 comprise a bearing outer race 1532, a bearing inner race 1533, ball bearings 1531 and ball bearing retainer 1538. The bearing arrangement 1530 is effective in allowing the impeller 1511 to rotate within the housing 1512 whilst minimizing axial and radial movement of the impeller 1511 when the impeller 1511 is operated in water, air or another biologically inactive fluid.

However, the conventional bearing arrangement 1530 has proven to be less effective when operated in blood. In a blood contacting environment where blood can access the bearing arrangement 1530 the bearing operates without difficulty for short periods of time. However, blood contact with the bearing components induces clot formation and these clots develop a tougher constituency (more organized) over time and bearing friction gradually increases until the clot becomes sufficiently organized to cause a stall of the motor driving the impeller.

As a result, it is not possible to operate a bearing arrangement 1530 that comprises ball bearings in contact with blood without the current (power) required to drive the micromotor 1541 (not shown) increasing over time. Further, when the impeller of the pump 1501 is driven by a micromotor a stall becomes likely or almost certain after a number of hours of operation.

For intravascular catheter blood pumps 1500 it is desirable that the impeller 1511 be driven by a small motor, referred to herein as a micromotor, for a number of reasons. Firstly, these blood pumping devices are minimally invasive devices and smaller devices are preferred in minimally invasive surgeries. Secondly, it is not desirable to use any more current (power) than is necessary to drive the pumping element (or impeller). While a larger and more powerful motor might be able to turn the pumping element, notwithstanding the formation of clot and resulting friction build up, it makes failure modes potentially more serious. These larger motors are strong enough to damage catheter components before failing which could result in patient harm.

Micromotors on the other hand operate at high speed with minimal current. As a result, when there is resistance in the drive system (that turns the pumping element) these micromotors stall without deflecting or damaging any catheter components and so they can do no harm to the patient.

It is an object of this invention to provide bearing arrangements for intravascular blood pumps that allow micromotors to operate pumping elements thereof at high speed and with low power consumption. Micromotors shall mean motors with a diameter of less than 10 mm and that operate at high speeds (10,000 to 100,000 RPMs) and typically use less than 2 amp of current during normal operation.

With reference to FIG. 1b, after a conventional ball bearing arrangement in direct contact with blood failed to deliver smooth operation the inventors attempted to isolate the conventional ball bearing arrangement from blood contact during operation by sealing the distal housing 1521 within which the bearing arrangement 1530 operates. The second approach taken to overcoming the clotting challenges was to exclude blood from the bearing arrangement 1530 completely such that the biologically active components of blood cannot interact with the elements of the bearing arrangement 1530.

In this variation, and referring to FIG. 1b, the distal housing 1521 is completely sealed from fluid ingress during operation. The distal cap 1535 is sealingly coupled to the distal end of the distal housing 1521 and the proximal region of the distal housing 1521 comprises a bearing seal assembly 1560. The bearing shaft 1513 extends through the bearing seal assembly 1560. The pump assembly may comprise a pump assembly tip 1514.

The blood pump 1500 of FIG. 1b is identical to that of FIG. 1a except that it includes the bearing seal assembly 1560. The bearing seal assembly 1560 comprises a sealing gland 1561, a seal spring 1562 and a seal locking ring 1563. The bearing seal assembly 1560 is configured to seal against the wall of the distal housing 1521 with its outer surface and to seal against the surface of the bearing shaft 1513 with the seal gland 1561. The seal spring 1562 controls the amount of force that is applied by the gland and onto the sealing surface of the bearing shaft 1531. The sealing gland 1561 was polyimide-filled PTFE material and provided low friction and low heat generation.

It will be noted that the contact pressure on the seal surface 1564 is key to the seal function. While high contact pressure ensures a better seal it also accelerates the rate of seal wear. Whereas at low contact pressure the seal gland 1561 may not maintain a seal with the bearing drive shaft 1513 but also shows slower wearing properties. The contact pressure is also related to the dimensions of the bearing drive shaft 1513 and seal assembly 1560 dimensions as well as the stiffness of the seal spring 1562.

The bearing arrangement 1530 comprises two ball bearing assemblies spaced apart in the distal housing 1521. Spacing the ball bearing assemblies apart increases the length over which the bearing shaft 1513 is supported by the bearing arrangement 1530 and this improves the concentricity with which the impeller 1512 can be held. This in turn is important in terms of reducing hemolysis and thrombosis within the pump. In the embodiment shown, the two bearing assemblies are spaced apart by using a bearing outer race 1532 as a spacer.

FIG. 1c shows an enlarged cross-sectional view of the bearing arrangement 1530 and bearing seal assembly 1560 inside the distal housing 1521 with the distal cap 1535 removed. The distal cap 1535 and the distal housing 1521 are configured to be coupled together such that there is no step in the outer surface of the distal housing 1521. The distal cap 1535 and the distal housing 1521 are sealing coupled together and provide a complete seal on the distal end of the bearing arrangement 1530.

FIG. 1d shows an end photo of bearing arrangement 1530 inside the pump housing 1511 with the distal housing cap 1535 removed. The bearing arrangement 1530 comprises a bearing outer race 1532, bearing inner race 1533, ball bearings 1531 and ball bearing retainer 1546 to hold the ball bearings spaced apart generally equiangularly.

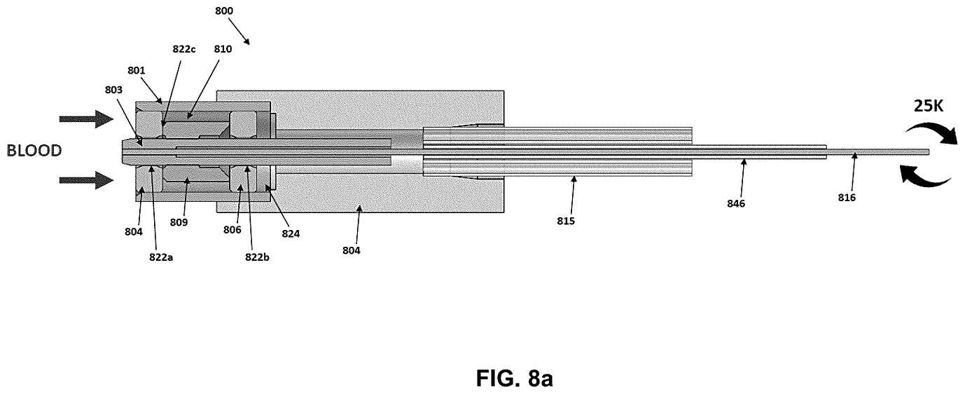

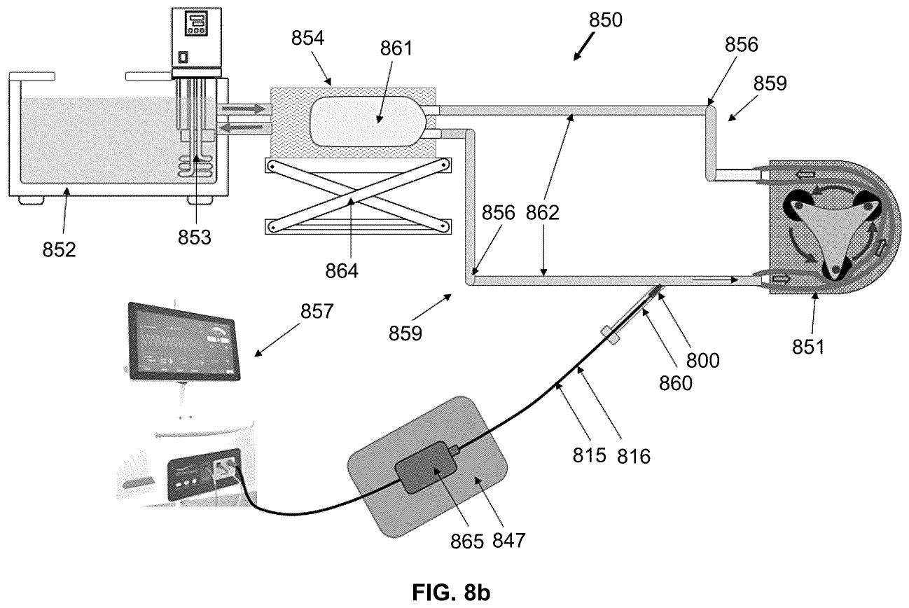

Catheter blood pumps 1500 comprising the bearing arrangement 1530 and bearing seal assembly 1560 of FIG. 1b through FIG. 1d were evaluated in a blood test model 850 of the type shown in FIG. 8 and the performance of the catheter blood pumps 1500 will now be discussed with reference to FIG. 1e through to FIG. 1h.

FIG. 1d shows a photo of a sample bearing shaft 1513a before assembly into the blood pump catheter 1500. It will be noted that the bearing shaft 1513a was manufactured with precision and comprises a highly polished surface.

FIG. 1f shows a photo of the of a bearing shaft 1513b after being tested in the blood flow model 850. The following can be observed in the picture: (i) discoloration 1537a of the bearing shaft 1513 at a proximal end of the sealing surface 1564, (ii) at least one pit mark 1537b on the bearing shaft 1513, (iii) deep grooves 1537c worn into the bearing shaft 1513 from the sliding interaction between the seal gland 1561 and the sealing surface 1564. It is also noteworthy that distal of the sealing surface 1564 the bearing shaft 1513 is blemish free 1537d.

FIG. 1g shows an end photo of bearing arrangement 1530 inside the pump housing 1511 with the distal housing cap 1535 removed after testing in the blood flow model 850. It can be seen that the bearing arrangement 1530 is covered with clot 1571 and this arose from the breakdown at the sealing surface 1564.

FIG. 1h shows a chart 1580 of the current 1582 used by the micromotor 1541 as a function of time while the pump assembly 1501 was being evaluated in the blood flow model 850 at a speed 1581 which was held constant at 25,000 revolutions per minute for the test. The test was operated for 15 hours in blood which was freshly drawn from a sheep prior to the test. The current 1582 used by the micro motor is charted relative to the time axis in hours. It can be seen in the chart 1580 that the current is very constant at just over 100 mA from time zero to 6 hours. At approximately 7 hours the first current instability 1584a, where the current spikes to approximately 160 mA, is seen. The second current instability 1584b occurs at between 10 hours and 10.5 hours. The second current instability 1584b lasts for close to 30 minutes and results in a current spike of 360 mA. The third current instability 1584c occurs at between 12.5 hours and 13 hours and this is stronger again and leads to a current spike of 600 mA which was sufficient to stall 1585 the motor.

The examples illustrated above highlight problems with conventional bearings and it is an object of this invention to solve these issues as well as to disclose newly discovered principles on how to optimize bearing arrangements for blood pumping devices.

### Novel Bearing Arrangements and Principles of Design

Specific embodiments of the present invention are now described in detail with reference to the figures, wherein the reference numbers indicate identical or functionally similar elements. The terms “distal” or “proximal” are used in the following description with respect to a position or direction relative to the treating physician. “Distal” or “distally” are a position distant from or in a direction away from the physician. “Proximal” or “proximally” or “proximate” are a position near or in a direction toward the physician.

The following detailed description is merely exemplary in nature and is not intended to limit the invention or the application and uses of the invention. In the present disclosure, the singular forms “a”, “an” and “the” includes the plural reference. It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such combinations are considered to be another embodiment. Conversely, various features of the invention that are, for brevity, described in the context of a single embodiment, may also be provided separately or in any sub-combination.

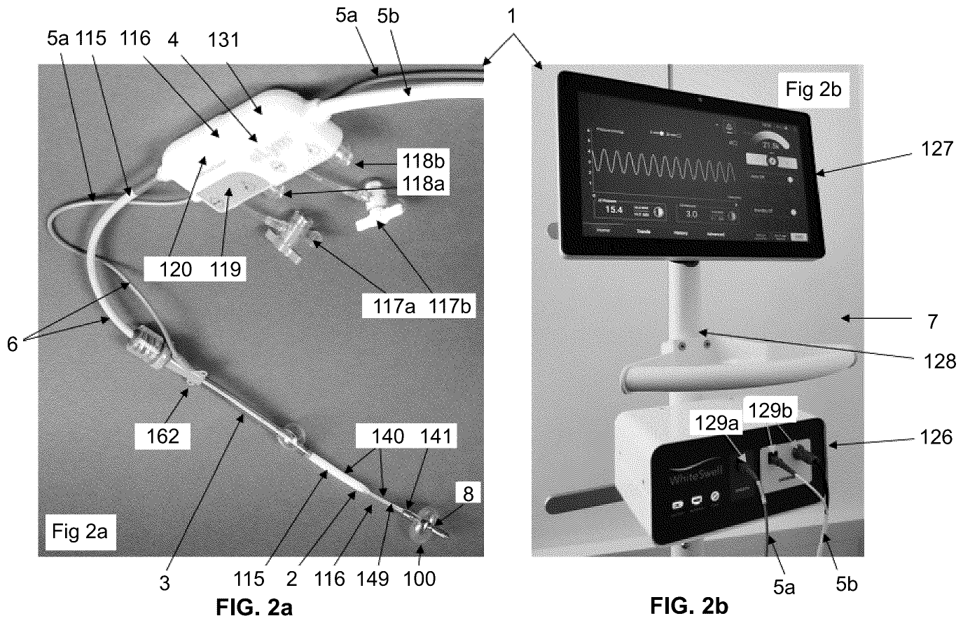

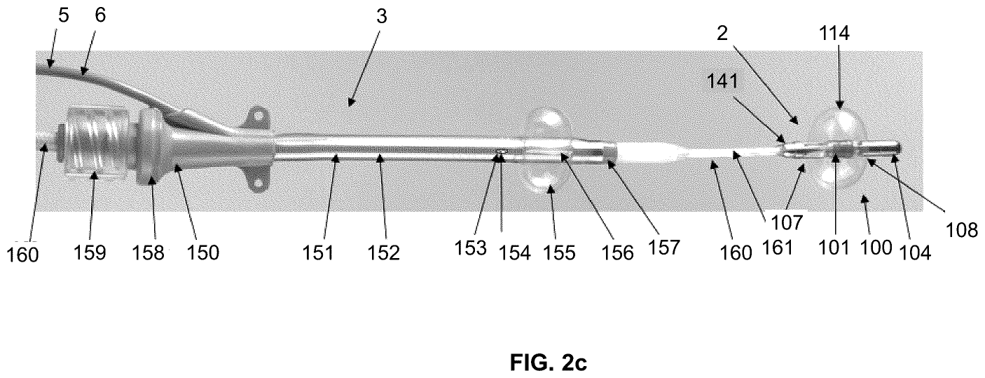

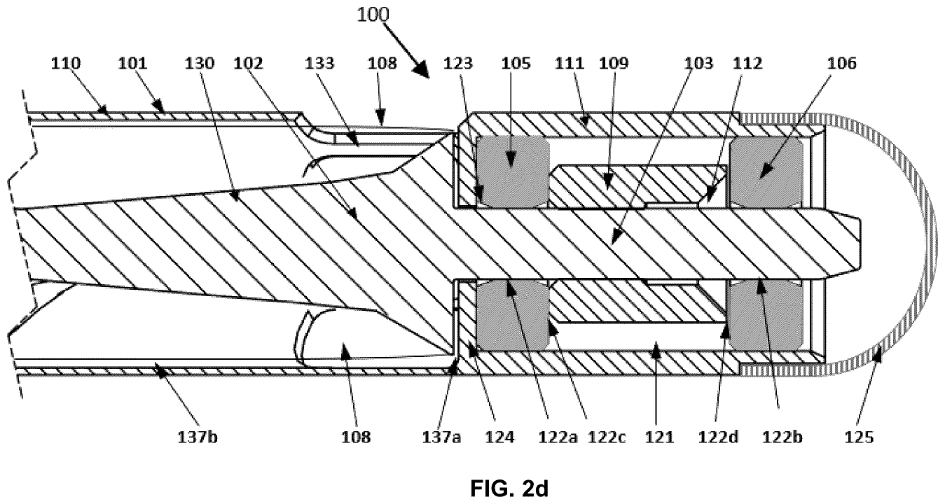

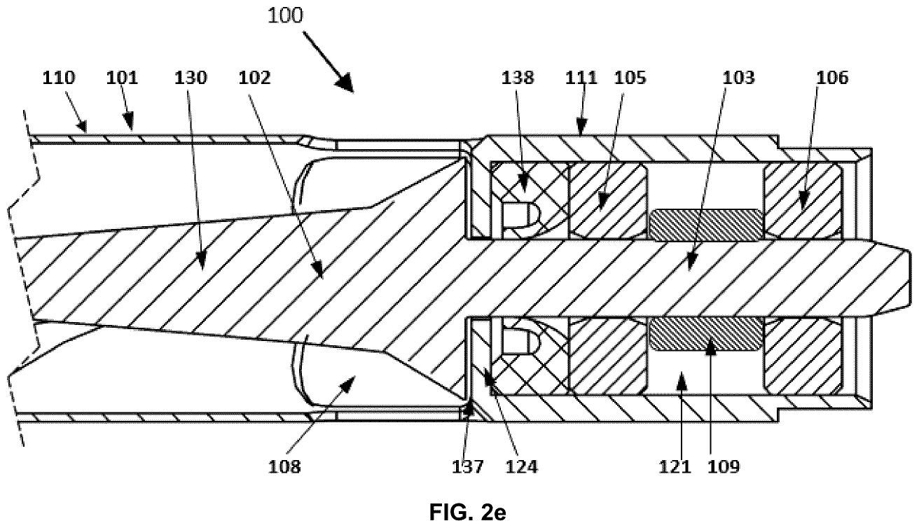

FIGS. 2a, 2b, 2c and 2d are schematic illustrations of an intravascular catheter system 1, in accordance with the respective embodiments of the present invention. The intravascular catheter system 1 is configured with components that operate inside the patient and in contact with the patient's bodily fluids (especially blood and lymph fluid) and components that are external of the patient and configured to control the operation of the internal components.

In an embodiment, the intravascular catheter system 1 comprises a blood pump 2, a sheath 3, a manifold 4, connection cables 5, and a console 7. The console 7 and the manifold 4 remain exterior of the patient during the procedure. At least portions of the sheath 3 and blood pump 2 are inserted into and operate within the patient (hereinafter referred as “indwelling components”). The console 7 is connected to the indwelling components and is configured to control the operation of the indwelling components and/or to support the operation of the indwelling components.

The console 7 is configured to receive information from the indwelling components and from the user operating the indwelling components to effect the treatment. The blood pump 2 and sheath 3 are configured to provide information to the console 7 and/or the user to allow the processing of information so as to manage and enhance the therapy. The information provided by the console 7 may comprise electronic information, pressure information, electrical information, impedance information, tactile feedback, and visualization information (via ultrasound or fluoroscopy).

The blood pump 2 and sheath 3 in the assembled configuration are depicted in FIG. 2a but without the blood vessel being shown. The blood pump 2 is configured such that its distal end can pass through the lumen of sheath 3 and into the blood vessel. The blood pump 2 comprises a pump assembly 100, a connecting cuff 141, a catheter shaft 140, a drive shaft 116, catheter connection cabling 5a, and a catheter manifold 120.

In an embodiment, the pump assembly 100 is at a distal region of the blood pump 2. The pump assembly 100 comprises a pump housing 101, a pumping element 8, a bearing arrangement 121, a pump assembly tip 104, a restrictor balloon 114, a pump inlet 107 and a pump outlet 108. The bearing arrangement 121 is configured to hold the pumping element 8, which rotates, inside the pump housing 101 while maintaining a precision gap 137 between pumping element 8 and adjacent static elements of the pump assembly 100. The pump assembly may include one or more static elements, i.e. elements that are at rest and/or are in equilibrium with their environment. The pump housing 101 may comprise one or more important static elements. The distal end of the cuff 141 may comprise another important static element. The housing separator 124 may comprise yet another important static element.

The bearing arrangement 121 holds the pumping element 8 inside the pump housing 101 such that a precision circumferential gap 137b is always maintained between the pumping element 8 and the inside diameter (ID) of the pump housing 101. The bearing arrangement 121 simultaneously holds the pumping element 8 inside the pump housing 101 such that precision axial gaps are maintained between the pumping element proximal and distal faces and the adjacent static surfaces. In one variation the pumping element 8 comprises an impeller pumping element (or impeller 102).

The bearing arrangement 121 is further configured to maintain two precision gaps at axial ends of the impeller 102. A distal impeller gap 137a is maintained by the bearing arrangement 121 between the distal face of the impeller 102 and the distal static surface.

In an embodiment, the distal static surface comprises a surface that is integral with the pump housing 101. In another embodiment, the distal static surface comprises a surface of a static bearing 105. The proximal impeller gap 137c (not shown) is maintained between the impeller 102 and a distal face of the connecting cuff 141. The bearing arrangement 121 is configured to allow the impeller 102 to rotate without frictional resistance.

The connecting cuff 141 establishes an interconnection between the pump assembly 100 and the catheter shaft 140. The connecting cuff 141 comprises a proximal opening into which the distal end of the catheter shaft 140 is joined. The cuff 141 is also fixedly joined to the pump housing 101. The pump 2 comprises at least one lumen through which the drive shaft 116 extends.

In an embodiment, the cuff 141 comprises a tapering distal nose which extends into the pump housing 101 and opposes but does not abut the proximal face of the impeller 102. The tapering distal nose of the cuff 141 is configured to allow bodily fluid to enter the pump without turbulence or recirculation.

The catheter shaft 140 extends from the cuff 141 of the pump assembly 100 to the catheter manifold 120. The catheter shaft 140 comprises an elongate member configured to simultaneously fulfill multiple objectives of the system 1. The catheter shaft 140 is a reinforced composite structure and comprises a multi lumen tubing 160. A first lumen of the multilumen tubing 160 allows the distal restrictor balloon 114 to be inflated and deflated using the stopcock luer fittings 117 at the catheter manifold 120. The first lumen of the multilumen tubing 160 extends from the manifold 120 through the catheter shaft 140 and extends into fluid connection with the ID of the restrictor balloon 114. The catheter shaft 115 comprises an enlarged region proximal of the distal end of the catheter shaft 140 and said enlarged region is compressible and is configured to appose the ID of the sheath to prevent blood migrating into the lumen of the sheath and coagulating therein.

The drive shaft 116 is connected to the impeller 102 to transmit rotational energy to the impeller 102 to pump bodily fluids. The drive shaft 116 comprises a small diameter rod like shaft that extends from its connection to the impeller 102 through the catheter shaft 140 and into the catheter manifold 120. The drive shaft 116 is coupled to a micro motor 131 inside the catheter manifold 120.

The sheath connection cabling 5a extends from the sheath hub 150 through the sheath manifold 119 and the cable 5a also extends proximally of the sheath manifold 119. The sheath connection cable 5a comprises a plug-in connector 129a at its proximal end and this connector 129a is configured to be plugged into the console 7. The sheath connection cable 5a comprises an outer protective tube and plurality of internal functional elements.

In an embodiment, the sheath connection cable 5a comprises a sheath flushing lumen, a sheath balloon inflation lumen, a pressure sensing lumen, and a pressure sensing cable.

The catheter connection cable 5b comprises at least one plug-in connector 129b at its proximal end. The catheter connection cable 5b extends from the catheter manifold 120 to the plug-in connector 129b. The plug-in connector 129b may comprise a plurality of connectors 129b as depicted in FIG. 2b.

In an embodiment, the catheter cable 5b comprises an outer protective jacket, a pressure sensing cable, and a motor power cable.

In another embodiment, the catheter connection cable 5b also comprises a catheter restrictor balloon inflation lumen and a catheter flushing lumen.

The catheter manifold 120 comprises an outer housing and a functionally designed inner space. In one embodiment, the manifold 120 is configured to hold the impeller drive shaft 116 and the drive shaft of the motor 131 co-axially with each other.

In an embodiment, the manifold 120 is configured to dampen vibrations of the motor 131 during the operation of the motor 131.

In an embodiment, the manifold 120 is configured to soundproof the motor 131 during the operation.

In an embodiment, the manifold 120 is configured to allow connection between the balloon inflation lumen and a balloon inflation luer exterior of the manifold.

In an embodiment, the manifold 120 is configured to allow connection between the catheter flushing lumen and a flushing luer exterior of the manifold. The catheter flushing lumen is configured to allow the delivery of flush media to the proximal gap 137c and/or the distal gap 137a of the pump assembly 100.

The sheath 3 is configured to access a blood vessel of a patient and to maintain access to the blood vessel during the procedure. The sheath 3 comprises a sheath hub 150, sheath shaft 151, sheath tip 157, and a sheath restrictor 155 at a distal region of the sheath shaft 151. The sheath hub 150 comprises a septum valve 158, configured to allow devices to pass through the lumen of the sheath 3 without blood loss, a luer cap 159 and suture holes 162.

In an embodiment, the sheath shaft 151 comprises an outer jacket, a reinforcement 152, an inflation lumen and a pressure sensing lumen. The reinforcement 152 comprises a metallic reinforcement configured to allow the sheath shaft 151 to flex with ease while being resistant to buckling or kinking.

In an embodiment, the sheath shaft 151 comprises one or more control lumens 6. One of said control lumens 6 comprises a pressure sensing lumen said pressure sensing lumens comprising a port or inlet 154. In one embodiment the sheath shaft 151 comprises a pressure sensor 153 said pressure sensor configured to measure or sense vascular fluid pressure proximal of the sheath restrictor balloon 155. In one embodiment the sheath shaft 151 comprises a port 154, said port 154 configured to establish fluid communication between a pressure sensor 153 in the pressure sensing lumen of the sheath shaft 151 and bodily fluid proximal of the sheath restrictor 155.

The distal region of the sheath 3 comprises a sheath tip 157 and a sheath restrictor 155. The sheath restrictor 155 comprises a collapsed state and an expanded state and in the expanded state the sheath restrictor is configured to appose the vessel wall without occluding fluid flow in the vessel. The sheath restrictor 155 is thus configured to restrict but not occlude fluid flow in the vessel. The sheath restrictor 155 comprises at least one restrictor bead 156, said restrictor bead 156 configured to ensure an uneven radial expansion of the sheath balloon 155 and said uneven expansion leading to fluid flow channels between the vessel wall and the sheath restrictor 155 when the restrictor 155 is inflated into apposition with the vessel wall.

The manifold 4 comprises a sheath manifold 119 and a catheter manifold 120. The sheath manifold 119 receives connection cable 5a from the sheath hub 150 and provides a flushing luer 118a for flushing the sheath 3 with saline or other biological fluids. The sheath manifold 119 also comprises a stopcock with luer fittings 117a for inflating, maintaining inflation and deflating the sheath restrictor 155. The sheath manifold receives the pressure sensing cables from the sheath hub 150 and conveys them onward to the console 7.

The catheter manifold 120 is coupled to the proximal end of the catheter shaft 115. The catheter shaft 115 contains a drive shaft 116, pressure sensor cables, catheter distal balloon inflation lumen and a catheter flushing lumen. The catheter manifold 120 receives the drive shaft 116 and supports the drive shaft 116 connection to the motor 131. The catheter manifold 120 receives the pressure sensor cables and convers them onward to the console 7. The catheter manifold 120 provides a flushing luer 118b for maintaining a low volume flush to and through the catheter distal gap 137a and/or the catheter proximal gap 137c with biological fluids. The catheter manifold 120 also includes a stopcock with luer fittings 117b for inflating, maintaining inflation and deflating the catheter restrictor 114. In one variation the sheath manifold 119 and catheter manifold 120 can be joined, nested or snapped together.

The console 7 comprises a touch screen display 127, a controller 126 and a mobile cart 128. The controller 126 receives information from the catheter pressure sensors 149 and the sheath pressure sensor 153 and controls the speed of the motor 131 which in turn determines the rate at which bodily fluids are pumped by the pump assembly 100. The mobile cart 128 is configured for placement beside the bed of the patient and can positionally be adjusted to make controlling the therapy easy and comfortable for the physician.

As depicted in FIG. 2d, the pump housing 101 comprises a proximal pump housing 110 and a distal pump housing 111. The impeller 102 is housed within the proximal pump housing 110 and the bearing arrangement 121 is housed in the distal housing 111. The distal pump housing 110 comprises a reception space into which the bearing arrangement 121 is inserted and the open end of the distal pump housing 110 is closed with housing distal cap 125. The bearing arrangement 121 comprises the bearing shaft 103, first static bearing 105, second static bearing 106 and retaining ring 109. The bearing shaft 103 extends from the distal face of the impeller 102 and is coaxial with the axis of the impeller 102. The bearing arrangement 121 comprises multiple bearing surfaces 122. One or more bearing surfaces 122a, 122b keeps the impeller 102 rotating coaxial with the pump housing 101 thus maintaining the body gap 137b between the impeller 102 and the ID of the pump housing 101. One or more bearing surfaces 122c, 122d keeps the impeller 102 axially fixed as it rotates and maintains the distal gap 137a and the proximal gap 137c during impeller 102 operation. The bearing shaft 103 has two bearing surfaces with the first static bearing 105 and also with the second static bearing 106. The first static bearing 105 and the second static bearing 106 are spaced apart from each other to enhance the lateral stability of the impeller 102 in the pump housing 101.

In an embodiment, the first bearing 105 and the second bearing 106 are compression fitted to the ID of the distal housing 111.

In another embodiment, the first bearing 105 and the second bearing 106 are bonded to the distal housing 111. The ID of the first bearing 105 and the second bearing 106 is closely sized to the outside diameter (OD) of the bearing shaft 103 with a very slight tolerance gap of 0.02 mm+/−0.001 mm. The retainer ring 109 is coupled to the bearing shaft 103 between the first static bearing 105 and the second static bearing 106. The retainer ring 109 has a proximal bearing surface 122c with the distal side wall of the first static bearing 105 and a distal bearing surface 122d with the proximal side wall of the second static bearing 106.

The bearing arrangement 121 is configured such that the retainer ring 109 bears on only one surface at any time. Thus, if the retainer ring 109 bears on the first static bearing 105 then there will be a slight gap between the retainer ring 109 and the second static bearing 106. It is an object of the present invention to keep this gap as small as possible while simultaneously avoiding unnecessary friction from an interference between the retainer and the static bearings (105,106). When the impeller 102 is operating, it is designed to accelerate fluid entering the pump assembly both axially and radially. This creates a reaction force that pushes the impeller 102 proximally. As a result, the bearing surface 122c between the first static bearing 105 and the retainer ring 109 will bear an axial load much more often that the distal bearing surface 122d and requires better wear properties than the surfaces of the distal bearing surface 122d. The distal end of the bearing shaft 103 comprises a smooth tapered end such that the first static bearing 105, the retainer ring 109 and the second static bearing 106 can be mounted on the bearing shaft 103 without damage or deformation to any of the components. Similarly, the reception space of the distal housing 111 comprises a smooth bore with a tapered entrance such that the first static bearing 105 and the second static bearing 106 can be advanced into the reception space without damage or deformation.

The bearings are made of suitable material, which is biocompatible, wear resistant, has good dimensional stability, and can be processed to precise dimensions. Preferably, the biocompatible material includes Ceramics such as Silica Nitride (Si3N4) and Zirconium dioxide (ZrO2), Corundums such as Ruby and Sapphire (Al2O3) or synthetic versions thereof, Metals such as Steel, Titanium and Nitinol, and Polymers such as PEEK (Polyether ether ketone), PAI (Polyamide imide), PPS (Polyphenylenesulfide), POM (Polyoxymethylene) and PI (Polyimide) or composites thereof.

Ceramics and Corundums are very hard and dimensionally stable materials. When paired together in bearing applications these materials can provide wear rates of <0.1 μm per year. Ceramics can be shaped by compressing a ceramic powder into its final shape before sintering to reduce the number of process steps. For precision bearing applications micron range dimensional and geometric tolerances can be achieved with additional machining processes. Hardened martensitic steels (with >0.4% Carbon) such as 440C stainless, 420F stainless and Sandvik 20 AP, or 20 WIV can be paired with Ceramics or Corundums in pivot or sliding bearing designs. Rockwell hardness should be a minimum of RC45 for such pairings. Suitable alloys include chrome, cobalt, and vanadium alloys such as Elgiloy N-100. Other materials in use are tungsten carbide which is a chemical compound containing equal parts of tungsten and carbon atoms offering similar hardness to Corundums.

It is important to appropriately match bearing materials for optimum performance based on the intended duration of use. A matched pair of bearing materials are materials that perform exceptionally well when sliding over each other for long periods of time in a bearing arrangement. For example, when PEEK is paired with Titanium wear does not becomes significant until time periods greater than one week. On the other hand, when a Corundum or Ceramic is paired with Titanium wear is significant in less than one day. When Corundums and Ceramics are paired with each other significant wear is not apparent in time periods of a year. If it is desirable to pair an unmatched pair of bearing materials due to manufacturing or design constraints Diamond Like Carbon (DLC) or Polycrystalline Diamond (PCD) coatings can be utilized to improve the wear performance of the pair. DLC is a high hardness and low friction coating with excellent hemocompatibility that is commonly used in medical device applications. Amorphous hydrogenated carbon a-C:H is a popular form of DLC coating that can achieve a Vickers hardness of 3500 Hv and a low coefficient of friction against steel of 0.1.

For reference, the minimum Rockwell hardness recommended above of RC45 equates to approximately 460 Hv. The hardest, strongest, and slickest form of DLC is tetrahedral amorphous carbon (ta-C) which consists almost entirely of sp3 bonded carbon atoms. Fillers such as hydrogen, graphitic sp2 carbon, and metals are used in the other forms to reduce production expenses or to impart other desirable properties.

The table 1 below shows matched pairs of bearing materials in accordance with the present invention:

TABLE 1
List of matched pairs of bearing materials

| Bearing | Bearing |
|---|---|
| Material #1 | Material #2 |
| PEEK | Stainless Steel |
| PEEK | Titanium |
| PEEK | Diamond Like Carbon |
| PEEK | Nitinol |
| PEEK | PEEK |
| PEEK Composite | Stainless Steel |
| PEEK Composite | Titanium |
| PEEK Composite | Diamond Like Carbon |
| PEEK Composite | Nitinol |
| PEEK Composite | PEEK Composite |
| PEEK Composite | PEEK |
| Diamond like Carbon | Diamond like Carbon |
| Ceramic/Corundum | Ceramic/Corundum |
| Ceramic/Corundum | Tungsten Carbide |
| Ceramic/Corundum | Stainless Steel |
| Ceramic/Corundum | Diamond Like Carbon |

FIG. 2e illustrates an alternative embodiment of the bearing arrangement 100 as described in FIG. 2a-d. In this embodiment, the bearing arrangement comprises a bearing shaft 103, first static bearing 105, second static bearing 106, retainer ring 109 and a bearing seal 138. The bearing seal 138 sits at the proximal end of the distal housing 11 and provides a sealing surface with the distal pump housing 111 and with the bearing shaft 103. The seal with the distal pump housing 111 comprises a static seal in that neither the seal 138 nor the distal housing 111 are moving with respect to each other. The seal between the bearing shaft 103 and the bearing seal 138 is a dynamic seal in that, in operation, the bearing shaft 103 rotates with respect to the bearing seal 138.

The bearing seal 138 incorporates a sealing lip 113 that is configured to be an interference fit over the bearing shaft 103. The degree of interference determines the contact pressure between the lip 113 and the shaft 103. A diametrical interference of between 5% and 15% is desirable. If contact pressure is too high accelerated seal wear can occur and if contact pressure is too low a seal may not be maintained. Material selection can also impact on contact pressure. A favorable material combination for the seal 138 is PTFE with Polyimide filler which achieves low friction from the PTFE component while wear resistance is imparted by the Polyimide filler. With these materials plastic deformation of the seal 138 can occur after insertion over the shaft 103. In an embodiment, this can be counteracted by incorporating a spring into the seal 138 over the lip 113 to better control contact pressure between the lip 113 and the shaft 103. In another embodiment, this effect can be counteracted by using a fluoroelastomer such as FKM with elastically deforms over the shaft 103 to create a seal. PTFE can be compounded with the fluoroelastomer to lessen friction in this embodiment. In this configuration a diametrical interference of between 10% and 20% is desirable.

The impeller 102 is shown in the proximal housing 110 and only the pump outlets 108 with outlet struts 133 are shown in the figure.

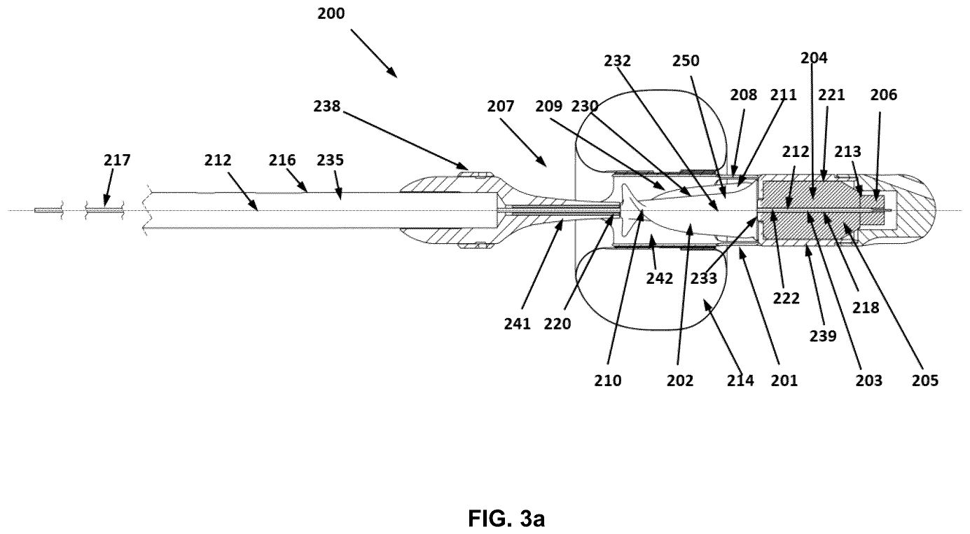

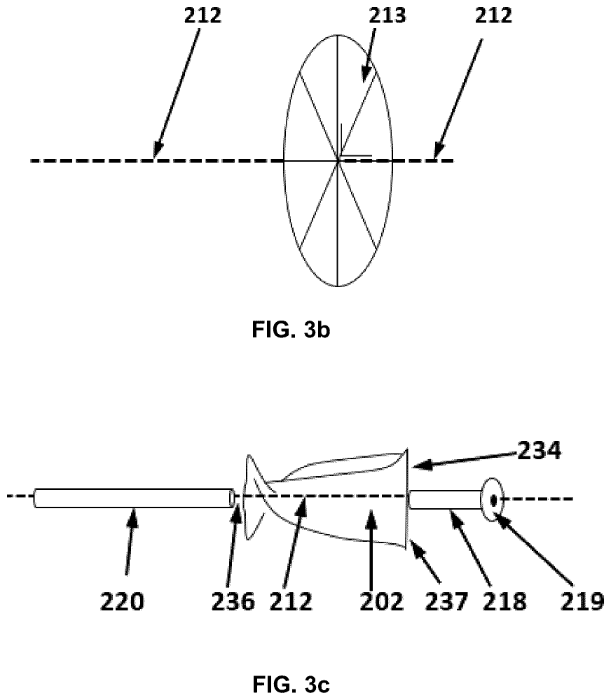

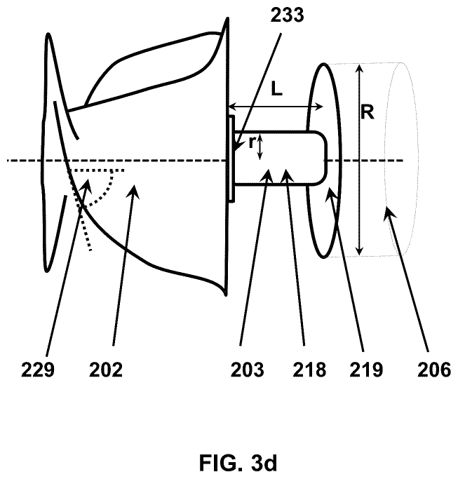

FIG. 3a schematically illustrates an intravascular blood pump assembly 200 of a catheter system in accordance with an embodiment of the present invention. In FIG. 3a, the pumping element 250 and the bearing arrangement 221 are highlighted with respect to other components of the pump assembly 200 and with the proximal portions of the catheter system stripped away. In FIG. 3a the pumping element 250 comprises an impeller 202. With this embodiment, the impeller 202 is restrained by three bearing surfaces. The first bearing surface 218 is defined by the bearing interactions of the bearing block 205 and the bearing shaft 203 as described in FIG. 3a. The second bearing surface 219 is defined by the bearing interactions of the bearing block 205 distal end and the proximal face of the retainer ring 206 as described in FIG. 3a.

The third bearing surface is the proximal bearing surface 220 and this bearing surface is defined by the bearing interactions of the drive shaft 217 and the ID of the cuff 241 adjacent to the impeller 202 proximal end.

In an embodiment, the intravascular blood pump assembly 200 comprises an elongate tubular housing 201 with at least one inlet 207 and at least one outlet 208, a proximal end and a distal end, an impeller 202 disposed within the elongate housing 201 at least partially between the at least one inlet 207 and the at least one outlet 208 and configured to rotate relative to the elongate tubular housing 201. The intravascular blood pump assembly 200 further comprises a bearing arrangement 221 adjacent the impeller 202 the bearing arrangement 221 comprising a plurality of bearing surface 222, at least one of said plurality of bearing surface 222 configured geometrically to generate a low bearing interfacial resistive force 228 when operated in a blood or plasma protein contacting environment. At least one of the plurality of bearing surfaces 222 of the bearing arrangement 221 comprises a geometric drag factor and the geometry of the bearing surfaces 222 being configured so as to generate a low geometric drag factor.

In another variation, at least one bearing surface 222 comprises a first bearing surface 218 and a second bearing surface 219 and first and second bearing surfaces comprise a continuous surface.

In an embodiment, the bearing arrangement 221 comprises a plurality of bearing surfaces 222 and a geometric drag factor. The geometric arrangement of the plurality of bearing surfaces 222 is configured so as to generate a low cumulative geometric drag factor.

In an embodiment, at least one of the bearing surfaces 222 comprises a static surface and a rotating surface and the surface area of the static surface and the rotating surface of the bearing arrangement 221 are substantially equal.

In another embodiment, the bearing arrangement comprises at least one bearing gap 227, the bearing gap 227 comprising a small space between a static surface and the rotating surface of the bearing arrangement 221.

In an embodiment, the intravascular blood pump assembly 200 comprises a bearing arrangement 221, which has been optimized and comprises a geometric drag factor for the bearing surfaces 222 of the bearing arrangement 221 of less than 4.0 mm³.

Preferably, the geometric drag factor for the bearing surfaces 222 of the bearing arrangement 221 of less than 2.0 mm³.

Preferably, the geometric drag factor for the bearing surfaces 222 of the bearing arrangement 221 is less than 1.0 mm³.

More preferably, the geometric drag factor for the bearing surfaces 222 of the bearing arrangement 221 is less than 0.5 mm³.

More preferably, the geometric drag factor for the bearing surfaces 222 of the bearing arrangement 221 is less than 0.2 mm³.

It will be appreciated that while optimizing a bearing arrangement for geometric drag factor (GF) can greatly improve the performance of a bearing in contact with blood, it is not the only factor. Other influencing factors include the bearing gap size, the biocompatibility of the bearing surfaces, the coefficient of friction of the bearing surfaces, the non-stick properties of the bearing surfaces.

In one embodiment, the bearing arrangement 221 is configured to generate a low bearing interfacial resistive force 228 and the low bearing interfacial resistive force 228 of the bearing arrangement 221 results partially from a low geometric drag factor of the at least one bearing surfaces 222.

The bearing interfacial resistive force of a bearing arrangement of the invention is a force that resists the rotational operation of the rotating components of the bearing arrangement (and thus the impeller) when in contact with biological fluids and especially blood. Surprisingly, the magnitude of the bearing interfacial resistive force increases with increasing geometric drag factor. It also surprisingly turns out that it is the bearing surfaces of the bearing arrangement, where the components of biological fluids congeal and retard rotation, that are the dominant contributor to bearing interfacial resistive force.

Still referring to FIG. 3a, the impeller 202 of the intravascular blood pump assembly 200 comprises a pair of radially opposed impeller blades 209, the impeller blades 209 comprise an impeller blade proximal end 210 and an impeller blade distal end 211 the impeller blades 209 further comprise a blade angel 229 with respect to the axis of the impeller 202. Preferably, the blade angle 229 of the impeller 202 varies along the axial length of the impeller 202.

In an embodiment, the blade angle 229 of the impeller blade proximal end 210 is configured to accelerate blood generally axially and the axial acceleration of the blood by the impeller blade proximal end 210 is aided by the elongate tubular housing 201 which comprises an enclosed tubular housing along this region of the impeller blade 209.

In an embodiment, the blade angle 229 of the impeller blade distal end 211 is configured to accelerate blood generally radially and the radial acceleration of the blood by the impeller blade distal end 211 is aided by the elongate tubular housing 201 comprising a plurality of outlets 208 along this region of the impeller blade 209. The impeller 202 comprises an impeller core 230, the impeller core 230 comprises a generally cylindrical body. The impeller core 230 may be configured to progressively compress blood into a tighter annular space as it is propelled axially along the impeller core 230 by the impeller proximal blades 210.

The intravascular blood pump assembly 200 further comprises a drive shaft 217 and a motor 231, the motor 231 comprising an electric motor and configured to rotate at high speed and the drive shaft 217 extending between the motor 231 and the impeller 202. In one variation, the distal end of the drive shaft 217 extends into the impeller core 230 along the impeller neutral axis 232.

The intravascular blood pump assembly 200 further comprises a bearing shaft 203 distal of the impeller 202, the bearing shaft 203 comprising at least one of said at least one bearing surfaces 222.

In an embodiment, the bearing arrangement 221 comprises a bearing block 205, the bearing block 205 comprising a plurality of bearing surfaces. The bearing block 205 comprises a first distal bearing surface 218 configured to restrain the impeller bearing shaft 203 substantially fixed relative to the neutral axis 232 of the impeller 202. The first bearing surface 218 is configured in use to bear against the impeller bearing shaft 203. The bearing block 205 comprises a second bearing surface 219 the second bearing surface 219 configured to at least partially restrain the impeller 202 in a substantially fixed axial position relative to the bearing block 205. The second bearing surface 219 is configured when in use to, from time to time, bear against the proximal surface 213 of the retainer ring 206. The bearing block 205 comprises a third bearing surface 233. The third bearing surface 233 configured to at least partially restrain the impeller 202 in a substantially fixed axial position relative to the bearing block 205. The third bearing surface 233 is configured from time to time in use to bear against a portion of the impeller distal surface 234.

The third bearing surface 233 in the shown configuration comprises a protrusion from the bearing block 205 the protrusion extending through the wall that separates the proximal housing 238 from the distal housing 239 and defines the gap distal of the distal surface of the impeller 202. In an alternate embodiment, the distal surface of the impeller 202 comprises a protrusion close to its central axis that extends into contact with the third bearing surface 233 of the bearing block 205. In this embodiment the third bearing surface 233 is either touching or at least partially inside the distal housing 239.

In an embodiment, the intravascular blood pump assembly 200 is configured such that the smooth operation of the impeller 202 is facilitated by restraining the movement of the impeller 202 to one axis of restraint 212 and one plane of restraint 213, the one axis of restraint 212 and the one plane of restraint 213 always being orthogonal to each other. The intravascular blood pump assembly 200 is configured for intravascular delivery to a treatment location. The intravascular blood pump assembly 200 comprises a catheter shaft 216, the catheter shaft 216 extending exterior of the patient and being connected to the elongate tubular member 201, the catheter shaft 216 further comprising a plurality of lumens 235 for the operation of the pump assembly 200. The elongate tubular member 201 comprises an expandable and contractable restrictor 214 the restrictor 214 encircling the elongate tubular member 201. The expandable and collapsible restrictor 214 comprises a compliant balloon 215.

In a preferred embodiment, the expandable and collapsible restrictor 214 comprises an elastomeric biocompatible material. The restrictor 214 comprises a polyurethane, a PEBAX or a silicone material or a mixture, blend, copolymer, or composite thereof.

In an embodiment, the bearing arrangement 221 comprises a bearing surface 220 located on the proximal side of the impeller. The proximal bearing surface 220 provides further stability to the impeller 202. With this embodiment, the proximal bearing surface 220 is configured to restrain the drive shaft 217 adjacent to the impeller 202 proximal end coaxial with the neutral axis 232 of the pump housing 201 inner diameter. The proximal bearing surface 220 is further configured in use to bear against the drive shaft 217 during operation. The proximal bearing surface 220 may comprises a plurality of bearing surface segments disposed equiangularly around the drive shaft 217. The equiangular disposition of the bearing surface segments allows simultaneous restraint of the drive shaft 217 and flushing the proximal gap 236 between the impeller 202 and the distal end of the cuff 241.

The pump assembly 200 comprises a fluid channel 242, the fluid channel 242 comprising an annular channel between the inner diameter of the housing 201 and the impeller core 230. The impeller blades 209 sit in the fluid channel 242 and in operation exert pumping forces on fluids in the fluid channel 242. The impeller core 230 forms one boundary of the fluid channel 242 and the inner surface of the housing 201 forms another boundary of the fluid channel 242. In one embodiment the distal region of the cuff 241 comprises another boundary of the flow channel 242. Bodily fluid enters the fluid channel 242 via the at least one pump inlet 207 at low velocity and the fluid is accelerated by the action of the impeller 202 as it progresses along the fluid channel 242 and it exits the fluid channel 242 and the pump 200 via the at least one pump outlet 208 at higher velocity relative to the velocity at the inlet 207.

The pump assembly 221 comprises an impeller proximal gap 236 wherein the impeller proximal gap 236 comprises a flat annular ring shaped spacing between a proximal end of the impeller 202 and a distal end of the cuff 241. The impeller proximal gap 236 comprises an inner diameter, an outer diameter and a gap thickness.

In one embodiment the outer diameter of the impeller proximal gap 236 comprises the outer diameter of the distal end of the cuff 241. In one embodiment the outer diameter of the impeller proximal gap 236 comprises the outer diameter of the proximal end of the impeller 202. The impeller 202 may comprise a proximal nose, the proximal nose comprising a generally cylindrical extension that extends proximal of the impeller blades 209. The diameter of the proximal end of the proximal nose may comprise the outer diameter of the impeller proximal gap 236 in this embodiment. In one embodiment the outer diameter of the drive shaft 217 may comprise the inner diameter of the impeller proximal gap 236.

In one embodiment the impeller proximal gap 236 comprises a “washer” shaped fluid reception space. Since the proximal end of the impeller 202 and a distal end of the cuff 241 comprise a transition between a static pump surface and a rotating pump surface the impeller proximal gap 236 is critical in preventing these surfaces from contacting each other in operation and scoring or otherwise damaging the surfaces in the impeller proximal gap 236. Furthermore, it is an objective of the bearing arrangements of the invention that the impeller proximal gap 236 is resistant to fibrin formation or clot formation in, at or adjacent to, the impeller proximal gap 236.

Flat or beveled or concave washer shaped gaps comprise favorable impeller proximal gap 236 of the invention with respect to resistance to fibrin formation or clot formation in, at or adjacent to, the impeller proximal gap 236. Configuring the bearing arrangement 221 such that fluid in the impeller proximal gap 236 experiences a shear rate of at least 1000 mm/mms in operation in blood is preferred in preventing clot or fibrin formation in the impeller proximal gap 236. Preferably fluid in the impeller proximal gap 236 experiences shear rate of at least 5,000 mm/mms in operation. Preferably fluid in the impeller proximal gap 236 experiences shear rate of at least 12,000 mm/mms in operation. Preferably fluid in the impeller proximal gap 236 experiences shear rate of at least 20,000 mm/mms in operation. In one embodiment fluid in the impeller proximal gap 236 experiences shear rate of 50,000 mm/mms while operating the pump at a high pump speed.

In one embodiment the impeller proximal gap 236 is configured such that in operation it exerts centrifugal force on biological fluids in the impeller proximal gap 236 such that fluid entering the impeller proximal gap 236 is expelled therefrom by said centrifugal forces thus creating a constant interchange of biological fluids in the impeller proximal gap 236.

The pump assembly 221 comprises an impeller distal gap 237. In one variation the impeller distal gap 237 comprises a flat annular ring shaped spacing between the distal end (or face) of the impeller 202 and a proximally facing surface of the tip. The impeller distal gap 237 comprises an inner diameter, an outer diameter and a gap thickness. In one variant the inner diameter of the impeller distal gap 237 comprises the diameter of the bearing shaft 203. In another variant the inner diameter of the impeller distal gap 237 comprises the outer diameter of the third distal bearing surface 233. The outer diameter of the impeller distal gap 237 comprises the diameter of the distal face of the impeller 202. The impeller distal gap 237 per this invention extends from the outer perimeter of the distal end of the impeller 202 inwardly. It will be appreciated that while the impeller distal gap 237 may extend inwardly to the bearing shaft 203 it may also extend only partially towards the bearing shaft 203. Indeed, FIG. 3a shows the distal bearing gap 237 extending inwardly from the outer perimeter of the impeller 202 but it terminates at the outer diameter of the third distal bearing surface 233 in this instance which is a substantially larger diameter than the diameter of the bearing shaft 203 in this embodiment. Indeed, the impeller distal gap 237 in this instance is a flat washer shaped gap.

The proximally facing surface of the tip may comprise an abutment surface internal of the housing 201, or a surface of the bearing 205 in the housing 201, or a surface of the bearing assembly 221 or another proximally facing static surface adjacent the impeller distal surface. Since the distal end of the impeller 202 and the proximally facing surface also comprise a transition between a static pump surface and a rotating pump surface the impeller distal gap 237 is critical in preventing these surfaces from contacting each other and scoring or otherwise damaging the surfaces in the impeller distal gap 237. Furthermore, it is an objective of the bearing arrangements of the invention that the impeller distal gap 237 is resistant to fibrin formation or clot formation. Configuring the pump assembly 200 such that fluid in the impeller distal gap 237 experiences a shear rate of at least 1,000 mm/mms in operation in blood is desirable in preventing clot or fibrin formation in the impeller distal gap 237. Preferably fluid in the impeller distal gap 237 experiences shear rate of 5,000 mm/mms in operation. Preferably fluid in the impeller distal gap 237 experiences shear rate of 12,000 mm/mms in operation. Preferably fluid in the impeller distal gap 237 experiences shear rate of 20,000 mm/mms in operation. In one embodiment fluid in the impeller distal gap 237 experiences shear rate of 80,000 mm/mms when operating at a high pump speed. In one embodiment fluid in the impeller distal gap 237 experiences shear rate of 160,000 mm/mms when operating at a very high pump speed.

In one embodiment the impeller distal gap 237 comprises an inner diameter and an outer diameter and the outer diameter of the impeller distal gap 237 comprises a boundary of the flow channel 242. Preferably fluids at the outer diameter of the impeller distal gap 237 experience in operation a shear rate of at least 20,000 mm/mms.

Shear rate is parameter that relates to flowing fluids. It measures the relative motion between adjacent layers of a moving liquid. Shear rate is the rate at which fluid layers move past each other in a fluid body. Shear rate is a measure of the velocity gradient in a body of fluid. When two flat surfaces move relative to each other with a fluid filling the space between the two fluids then the shear rate in the fluid is simply the ratio of: (i) the velocity of the moving surface, and (ii) the gap between the two surfaces. In the case of proximal impeller gap 236 and the distal impeller gap 237 the velocity at any point or radius in the gap is simply the circumference multiplied by the rotational speed while the gap is the gap dimensions at the given point or radius.

In general terms, shear rate is determined by both the geometry and speed of the flow and as stated above is defined by the formula:

Shear Rate=Velocity/Distance

Where Velocity is the speed of a first layer of fluid relative to the second layer of fluid and Distance is the distance between the two fluid layers.

In an embodiment, the restrictor 214 is disposed between the at least one inlet 207 and the at least one outlet 208. The restrictor 214 is at least partially disposed over the impeller 202. The restrictor 214 comprises a tapering funnel region on its proximal end and said tapering funnel region guides blood flow or bodily fluid flow into the inlet 207 of the elongate tubing 201 without fluid turbulence or recirculation.

It will be appreciated that the pump assembly 200 as illustrated FIG. 3 can be integrated with sheaths, manifolds, cabling and consoles as described elsewhere in this disclosure. The intravascular blood pump assembly 200 comprises an elongate tubular housing 201 with a proximal end 238 and a distal end 239. The proximal end 238 of the elongate tubular housing 201 comprises at least one inlet 207 and at least one outlet 208, an impeller 202 disposed within the elongate housing 201 at least partially between the at least one inlet 207 and the at least one outlet 208. The impeller 202 is configured to rotate relative to the elongate tubular housing 201.

The intravascular blood pump assembly 200 further comprises a bearing block 205 located in the distal housing 239, the bearing block 205 comprising a generally cylindrical body cylindrical body comprising a lumen configured to accommodate bearing shaft 203. The bearing block 205 further comprises a first bearing surface 218, a second bearing surface 219 and a third bearing surface 233. The first bearing surface 218 comprises the surface where the OD of the bearing shaft 203 bears on the ID of the bearing block 205.

In an embodiment, the first bearing surface extends the length of the cylindrical body of the bearing block 205.

In an embodiment, the first bearing surface 218 comprises one or more segments of the length of the bearing block 205.

The second bearing surface 219 comprises at least a portion of a distal surface of the bearing block 205.

In an embodiment, the second bearing surface 219 comprises at least partially an axially distally facing surface. In another variant, the second bearing surface 219 comprises an annular or disc shaped surface.

In one embodiment the first bearing surface 218 defines an axis of restraint 212, and the second bearing surface 219 defines a plane of restraint 213 wherein the axis of restraint 212 and the plane of restraint 213 are orthogonal to each other. The third bearing surface 233 comprises a proximal surface of the bearing block 205.

In an embodiment, the third bearing surface 233 comprises at least partially an axially proximally facing surface. In another variant, the third bearing surface 233 comprises an annular or disc shaped surface.

In one embodiment the intravascular catheter blood pump assembly 200 comprises a bearing block 205 and a retainer ring 206 wherein the bearing block 205 is configured to bear against the retainer ring 206. In this embodiment the retaining ring 206 may comprise a homopolymer of a bearing material and the bearing block 205 may comprise a composite. The composite may further comprise the homopolymer of the bearing material as at least one constituent. In one variation the homopolymer of the bearing block 205 comprises a PEEK, nylon, polyimide or polyacetal composite and the composite further comprising one or more of PTFE, Graphite, and Carbon fiber as composite fillers. Since an important principle of blood pump bearing design comprises the pairing of bearing materials it will be appreciated that a similar effect would be achieved if the bearing block 205 comprised the homopolymer and the retainer ring 206 comprised the composite.

In one variation the bearing block 205 comprises a PEEK composite loaded with carbon fibers to between 25 and 35%. In another variation the bearing block 205 is configured to bear against the impeller distal surface 234. The impeller distal surface may comprise a metal or it may comprise a homopolymer from the bearing materials of this patent. The impeller distal surface may comprise a stainless steel, titanium, nitinol or other biocompatible metal bearing surface. The impeller distal surface 234 may comprise a PEEK, nylon, polyimide or polyacetal bearing surface. In one embodiment the impeller distal bearing surface comprises a diamond-like-carbon bearing surface.

In another variation of this embodiment the bearing block 205 comprises a first bearing surface 218 that is configured to bear against the outer diameter of the bearing shaft 203, wherein the bearing shaft 203 is comprised of a metal and the bearing block 205 comprises a composite. In one embodiment the first bearing surface 218 comprises a through hole that extends entirely through the bearing block 205. Preferably the through hole of the first bearing surface 218 comprises precision hole whereby the axis of the through hole is precisely concentric with the axis of the bearing block 205 and the reception space 204 of the pump assembly 200.

FIG. 3b sets out the axes and planes of restraint that are desirable from the bearing arrangements 221 of the present invention. Preferably, the bearing arrangement 221 is configured to restrain the impeller 202 rotationally to a single axis of rotation 212 that is coaxial with the inside diameter of the pump housing 201. Preferably, the bearing arrangement 221 is configured to restrain the impeller 202 axially to a single plane of rotation 213 and said plane of rotation 213 comprises a disc shaped plane or an annular plane that is perpendicular to the single axis of rotation 212.

In an embodiment, the bearing arrangement 221 is configured so as to define for the impeller 202 an axis of impeller restraint 212 and a plane of impeller restraint 213. The axis of impeller restraint 212 is collinear with the central axis of the pump housing 201 and the plane of impeller restraint 213 wherein the plane of impeller restraint 213 comprises a discoid surface that is normal to the axis of impeller restraint 212.

In an embodiment, the discoid surface of the plane of restraint 213 comprises an annular surface. In another variant, the discoid surface of the plane of restraint 213 comprises a curved surface. In another variant, the discoid surface of the plane of restraint 213 comprises a surface configured to abut an opposing surface.

In an embodiment, the second bearing surface 219 comprises a cylindrical surface and the plane of restraint 213 comprises a plane radial to the axis of the elongate tubular housing 201. The inlet 207 to the pump assembly 200 is located distal of the outlet 208, and the inlet 207 and outlet 208 are spaced apart from each other.

In an embodiment, the second bearing surface 219 comprises a cylindrical surface and the plane of restraint 213 comprises a plane radial to the axis of the elongate tubular housing 201. The inlet 207 to the pump assembly 200 is located proximal of the outlet 208 and the inlet 207 and outlet 208 are spaced apart from each other.

The distal end 239 of the elongate tubular housing 201 comprises a bearing reception space 204, the bearing reception space 204 configured to house the bearing block 205 fixedly hold the central axis of the first bearing surface 218 co-axial with the axis of restraint 212 of the impeller 202. The bearing block 205 is mounted in a circumferential interference fit with a distal region 239 of the elongate tubular housing 201. The first bearing surface 218 and the second bearing surface 219, comprise low friction wear resistant materials.

The first bearing surface 218 and the second bearing surface 219, comprise one or more of a polyether ether ketone, titanium, cobalt chrome, graphite, polyimide or a blend, composite or combination of one or more of these materials.

In an embodiment, the intravascular blood pump assembly 200 comprises a first bearing gap 223 at the first bearing surface 218. The first bearing gap 223 comprising an annular gap and the thickness of the annulus is less than 0.010 mm.

FIG. 3c schematically illustrates the dimensions of restraint of the impeller 202 in accordance with an embodiment of the present invention. It will be appreciated that a poorly restrained impeller will vibrate, will have varying gap dimensions, will pump less efficiently, and may even contact and score surfaces adjacent to the impeller 202. On the other side, overly constraining the impeller 202 leads to excessive friction for the motor to overcome, increased heat buildup at the bearing surfaces and greater likelihood of device failures or poor durability.

FIG. 3d schematically illustrates the bearing surfaces of the bearing arrangement 221 of the invention. The bearing shaft 203 extends distal of the impeller 202 and is concentric therewith and the surface of the bearing shaft 203 comprises the first bearing surface 218. The bearing shaft 203 bears on the surface of the ID of the bearing block 205. The second bearing surface 219 of the bearing arrangement 221 comprises the proximally facing surface of the retaining ring 206. The proximally facing surface of the retaining ring 206 bears on the distally facing surface of the bearing block 205. The third bearing surface 233 comprises a portion of the distal face of the impeller 202. The distal face of the impeller 202 bears on a proximally facing surface of the bearing block 205. The third bearing surface 233 may be configured in several ways. As shown in FIG. 3a and FIG. 2d the pump assembly may comprise a separator between the impeller and the bearing assembly. In this scenario the bearing block 205 comprises a stepped down proximal end and the stepped down proximal end extends through a central orifice of the separator into bearing contact with a distal surface of the impeller 202. Alternatively the pump assembly may comprise no separator and in this alternative the proximal surface of the bearing block bears directly on the distally facing surface of the impeller 202. Preferably with this embodiment the distally facing surface of the impeller 202 or the proximally facing surface of the bearing block 205 is stepped so as to minimize the area of the third bearing surface 233.

The present invention discovered that blood interacts with bearing surfaces very differently as compared to other fluids. It is well known that blood will form thrombus when exposed to bearing surfaces through a combination of static, recirculating and shear induced mechanisms and that this clot impacts the performance of the bearing assembly by creating an additional viscous drag force that needs to be overcome by the motor. However, the present invention discovered that a geometric parameter of the bearing surfaces significantly influences the degree to which thrombosis within the bearing housing impacts the performance of the bearing. This geometric parameter, referred to in this patent as the Geometric Drag Factor (GF), depends only on the shape and dimensions of the bearing surfaces of the bearing arrangement. The Geometric Drag Factor depends on the bearing area of the bearing surfaces as well as the absolute distance of the bearing area from the axis of rotation of the bearing.

Geometric drag factor is defined as sum of all the incremental areas that make up the bearing surfaces multiplied by the absolute normal distance between each area increment and the axis of rotation of the bearing arrangement.

In mathematical terms the geometric drag factor (GF) is defined as:

$$GF = \int ri . da$$

Where:
ri=the absolute normal distance between da and the axis of rotation of the bearing arrangement.
da=a small portion of area of the bearing surface.

The limits of the integration are set by the specific geometric features of the bearing surfaces for which GF is being calculated. Where there are multiple bearing surfaces in a bearing arrangement, the GF for the bearing arrangement will comprise the sum of the individual GFs. The Geometric Drag Factor is a very powerful tool in the design of blood contacting pumps and allows various pump designs to be compared and optimized without need to conduct large and expensive animal studies.

The Geometric Drag Factor bears similarities to a parameter known as the first moment of area in the field of solid mechanics. However, it is different. The first moment of area of a shape about any reference axis is the product of the area of shape multiplied by the distance between the centroid of shape and the reference axis. With the first moment of area of a shape if the distance between the centroid of shape and the reference axis is zero then the first moment of area is zero. This does not happen for the geometric drag factor for blood contacting bearing arrangements.

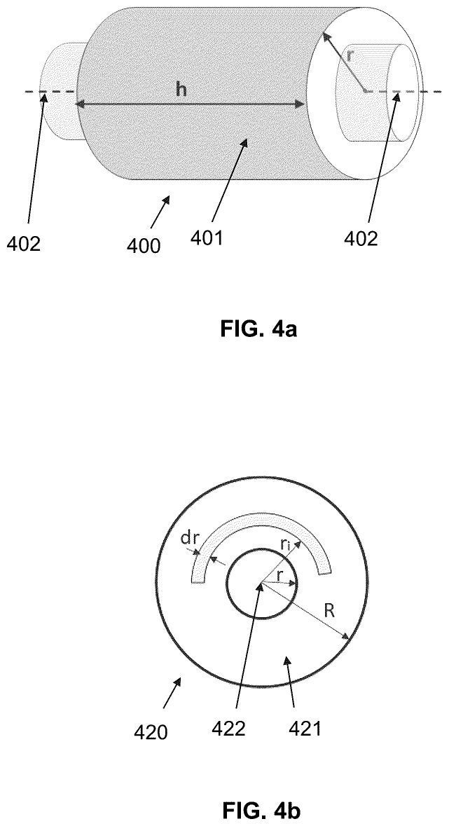

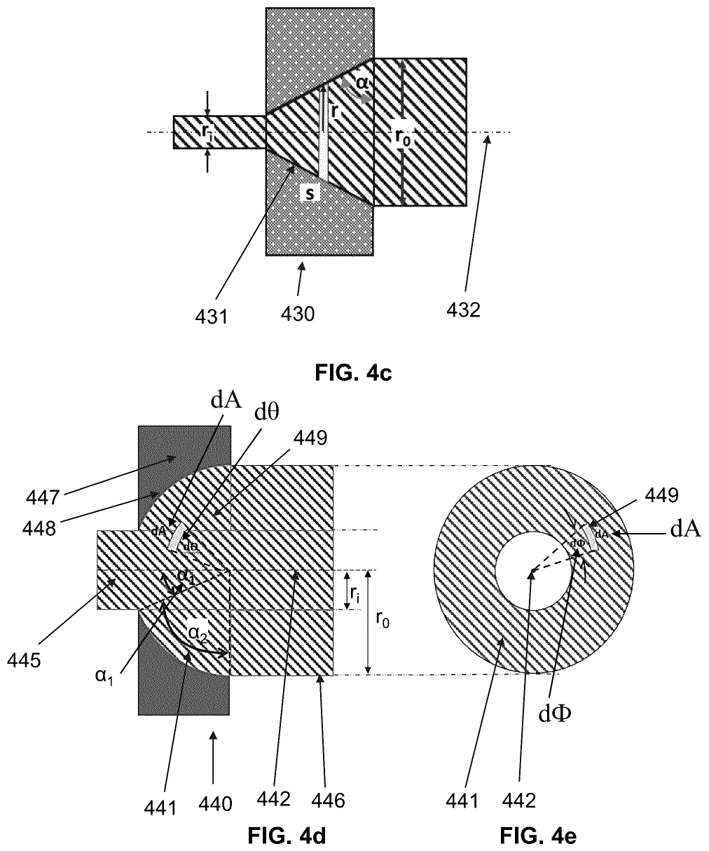

In FIG. 4a, a cylindrical bearing 400 is represented with a bearing surface 401 and an axis of rotation 402 about which the bearing surface rotates. The bearing surface 401 comprises a diameter of 2r and a length of h. The Geometric Drag Factor for this simple bearing surface is derived below:

$$GF = \int ri . da$$

Since each ri is the same distance (r) from the axis of rotation the GF is:

$$GF = r \times A , or \\ GF = π r 2 h \qquad Equation 1$$

Equation 1 allows the designer to calculate the contribution to the geometric drag factor for a bearing that is comprised at least partially of a cylindrical bearing surface.

In FIG. 4b, an annular bearing 420 is represented with a bearing surface 421 and an axis of rotation 422 about which the bearing surface rotates in operation. In this variant, the bearing surface 421 is an annular surface with an annulus inner radius of r₀ and an annulus outer radius of r₁. The area A of the annulus (A)=π(r₀²−r₁²), and, the area of Δa=2πr dr. Thus,

$$GF = \int r , dA \\ GF = {\int }_{{r}_{1}}^{{r}_{0}} 2 π {r}_{i}^{2} dr \\ GF = \frac{2 π}{3} ( {r}_{o}^{3} - {r}_{i}^{3} ) \qquad Equation 2$$

It will be appreciated that if the bearing surface 421 is a disc rather than an annulus that equation 2 can still be used and, in this situation, rᵢ=0. Equation 2 allows the designer to calculate the contribution to the geometric drag factor for a bearing that is comprised at least partially of an annular bearing surface.

In FIG. 4c, a conical or frustoconical bearing 430 is represented with a bearing surface 431 and an axis of rotation 432 about which the bearing surface rotates in operation. In this variant, the bearing surface 431 is in the shape of a frustrum. The length of the side of the frustrum is denoted “s” with a small end radius being denoted r1 and a large end radius being denoted r₀. The angle of the frustrum is also shown and denoted α in FIG. 4c. FIG. 4c also shows a small rim of area on the surface of the frustrum dA. Thus:

$$dA = 2 π rds \\ s = \frac{R}{cos α} \\ dA = \frac{2 π r}{cos α} dr \\ GF = \int \frac{2 π {r}^{2}}{cos α} dr \\ GF = \frac{2 π {R}^{3}}{3} ( \frac{1}{cos α} ) \qquad Equation 3$$

$$GF = \frac{2 π}{3} ( {r}_{o}^{3} - {r}_{i}^{3} ) ( \frac{1}{cos α} ) \qquad Equation 4$$

Equation 3 assumes that the bearing surface is a cone with R being the radius of the base of the cone. Equation 4 is a general form for a frustoconical bearing surface. If rᵢ is set at zero then equation 4 reduces back to equation 3. Equation 4 allows the designer to calculate the contribution to the geometric drag factor for a bearing that is comprised at least partially of a frustoconical bearing surface.

In FIGS. 4d and 4e, a spherical bearing arrangement 440 is illustrated with a bearing surface 441 and an axis of rotation 442 about which the bearing surface 441 rotates in operation. FIG. 4d schematically illustrates a cross sectional view of the bearing arrangement 440 while FIG. 4e schematically illustrates an end view of the bearing surface 441 only. In this variant, the bearing surface 441 comprises a portion of a sphere. The first shaft 445 and second shaft 446 extend from both ends of the bearing surface 441. Neither the first shaft 445 nor the second shaft 446 comprise bearing surfaces in this arrangement. The bearing surface 441 bears on an opposing surface 448 of the static bearing 447.

In solving for the Geometric Drag Factor in this variant, one must integrate between the limits of the bearing surface dimensions. In the cross-sectional view, the bearing surface 441 of the bearing arrangement 440 starts at angle α₁ and ends at angle α2 with respect to the centre of the sphere and the axis of rotation 442.

In the end view, the bearing surface 441 of the bearing arrangement is completely circular and so one integrates between 0 and 360°. It will be appreciated that the bearing surface 447 in end view, could comprises one or more segments of a spherical surface, and that a segmented spherical bearing surface would likely produce an improved GF and in this situation the limits of the second integration would be less than between 0 and 360°.

An increment of area 449 on the bearing surface 441 is denoted “dA”. dA is shown in both the cross-sectional view (FIG. 4d) and the end view (FIG. 4e). In FIG. 4d, the increment of area dA comprises an angle dΦ and in FIG. 4e, the increment of area dA comprises an angle dθ normal to dΦ.

The length of one side of the area increment ds1 is:

ds₁=R∂θ

The length of the other side of the area increment ds2 is:

ds₂=r∂Ø

The area dA is thus:

$$dA = {ds}_{1} {ds}_{2} = R \cdot {r}_{o} \cdot \partial θ \partial ∅ \\ {r}_{o} = R sin θ = > R = \frac{{r}_{o}}{sin θ} \\ dA = {ds}_{1} {ds}_{2} = \frac{{r}_{o}^{2}}{sin θ} \partial θ \partial ∅$$

Substituting dA into the general equation for GF and integrating:

$$dA = \frac{{r}^{2}}{sin θ} \partial θ \partial ∅ \\ GF = \int \int \frac{{r}^{3}}{sin θ} \partial θ \partial ∅ \\ lever arm : r = {r}_{0} sin θ \\ GF = {\int }_{0}^{2 π} {\int }_{{α}_{1}}^{{α}_{2}} ( {r}_{o}^{3} ) \cdot ( {sin}^{2} θ ) \partial ∅ \partial θ \\ GF = {\int }_{0}^{2 π} {\int }_{{α}_{1}}^{{α}_{2}} ( {r}_{o}^{3} ) \cdot ( {sin}^{2} θ ) \partial ∅ \partial θ \\ GF = 2 π ( {r}_{o}^{3} ) {\int }_{{α}_{1}}^{{α}_{2}} ( {sin}^{2} θ ) \partial θ \\ Since \int {sin}^{2} x = x / 2 - ( sin 2 x ) / 4 \\ GF = π [ {r}_{o}^{3} ] [ ( {α}_{2} - {α}_{1} ) + \frac{1}{2} ( ( sin 2 {α}_{1} ) - ( sin 2 {α}_{2} ) ) ] \qquad Equation 5$$

If if α₁=0, and α₂=90 then Equation 5 reduces to Equation 6.

$$GF = 2 π {r}_{o}^{3} \qquad Equation 6$$

Equation 5 and equation 6 allow the designer to calculate the contribution to the geometric drag factor for a bearing that is comprised at least partially of a spherical bearing surface.

It will be appreciated that a multitude of bearing geometries are possible but one skilled in the art will be able to apply the principles disclosed in these variants to calculate the GF for any blood contacting bearing arrangement and thus rapidly optimize the bearing design for interaction in a biologically active environment.

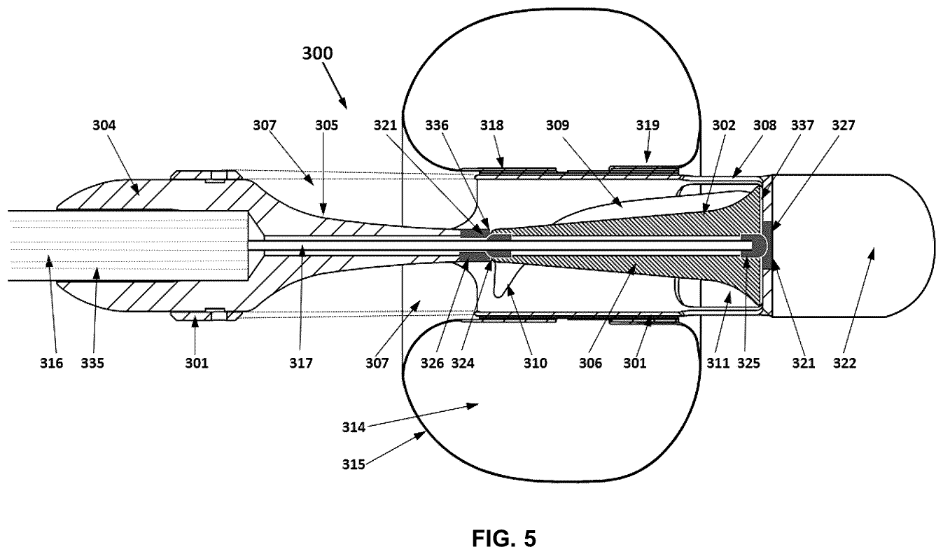

An alternative embodiment of a pump assembly 300 of intravascular catheter system 1, in accordance with the present invention is described in FIG. 5. The pump assembly 300 comprises a pump housing 301, an impeller 302, and a supporting cuff 304. The supporting cuff 304 of the pump assembly 300 is connected to a distal end of catheter shaft 316. In an embodiment, the distal end of the supporting cuff 304 comprises an abutment surface that engages with a stepped abutment surface of the impeller 302 and the cuff abutment surface supports in maintaining the axial position impeller 302. The supporting cuff 304 comprises a cuff transition region 305 which is configured to direct blood into the housing 601 without fluid turbulence or recirculation.

The pump assembly 300 comprises a catheter tip 322. The bearing arrangement 321 of the pump assembly 300 is not located as distally in the housing 301 as was the case with bearing arrangements shown in earlier figures. Instead, the bearing arrangement 321 comprises two bearing surfaces with the distal bearing surface immediately distal of the impeller 302 and a proximally bearing surface proximal of the impeller 302, both bearing surfaces within the housing 301. The bearing arrangement 321 comprises a proximal concave static bearing 326, a distal concave static bearing 327, a proximal ball nosed moving bearing 324 and a distal ball nosed moving bearing 325. The proximal concave static bearing 326 is fixedly coupled to the distal end of the supporting cuff 304 shaft and comprises an inner lumen configured to allow the drive shaft 317 to pass there through. The proximal concave static bearing 326 further comprises a spherical concave bearing surface the spherical concave bearing surface configured to bear against the proximal ball nosed moving bearing 324. The spherical concave bearing surface is at least partially spherical and includes a hard-wearing low friction material. The bearing surface of the proximal concave static bearing 326 extends from the drive shaft lumen through the proximal concave static bearing 326 to its distal most end.

The distal concave static bearing 327 comprises a ball nose shaped bearing surface at the proximal end of the catheter tip 322. The distal concave static bearing 327 sits inside a recess in the catheter tip 322 and comprises a hard-wearing low friction material. The proximal ball nosed moving bearing 324 comprises a proximally facing ball nosed projection of the impeller 302. The proximal ball nosed moving bearing 324 may be integral with the impeller 302 and its central axis is coaxial with the impeller 302. The proximal ball nosed moving bearing 324 comprises an inner lumen through which the drive shaft 317 passes and it is made from a hard-wearing low friction material.

In one embodiment, the proximal ball nosed moving bearing 324 is coupled to the drive shaft 317 with the proximal ball nosed moving bearing 324 in turn couples to the impeller 302. In alternative embodiment, the proximal ball nosed moving bearing 324 is coupled indirectly to the drive shaft 317. At least a portion of the proximally facing projection of the proximal ball nosed moving bearing 324 comprises a spherical bearing surface that is configured to bear on the bearing surface of the of the proximal concave static bearing 326. Preferably the concavity of the proximal ball nosed moving bearing 324 matches the concavity proximal concave static bearing 326. Similarly, and preferably the concavity of the distal ball nosed moving bearing 325 matches the concavity of the distal concave static bearing 327.

Preferably the bearing arrangement 321 is configured to simultaneously limit the range of movement of the impeller 302 in the axial direction and in the radial direction. The proximal concave static bearing 326, the distal concave static bearing 327, the proximal ball nosed moving bearing 324 and the distal ball nosed moving bearing 325 are configured to limit the range of movement of the impeller 302 to 20 microns or less in the axial direction and 30 microns or less in the radial dimension. These movement limits mean that the proximal and distal bearing surfaces of the bearing arrangement 321 are bearing surfaces as defined in accordance with the present invention.

It will be appreciated that the bearing surfaces of the concave bearings 324, 325, 326 and 327 could be conical, frustoconical, cylindrical or generally tapered bearing surfaces and these alternate bearing arrangements are configured to simultaneously limit the range of movement of the impeller 302 in the axial direction and in the radial direction.

In an embodiment, the proximal concave static bearing 326 comprises a first bearing material and the proximal ball nosed moving bearing 324 comprise a second bearing material and said first bearing material and said second bearing material comprise a matched pair of bearing materials as described elsewhere in this patent. Similarly the static concave static bearing 327 and distal ball nosed moving bearing 325 also constitute a bearing pair.

In one embodiment a first part of a bearing pair may comprise a homopolymer and the second a composite, the composite may further comprise the homopolymer of the first part as at least one constituent. In one variation the homopolymer comprises a PEEK, nylon, polyimide or polyacetal and the composite comprises one of said homopolymers and further comprises one or more of PTFE, Graphite, and Carbon fiber as composite fillers. In another embodiment the arrangement of the bearing pair may be in an opposite configuration and still maintain favorable performance. In other variations suitable bearing pairs from the materials listed in Table 1 can be substituted.

For the purpose of this invention a “bearing” is a mechanical arrangement of a blood pump that limits relative motion of the operable blood pump elements to only the desired motion (to pump blood) and reduces friction between moving parts of the blood pump.

Bearings of the invention comprise a static element(s) and a moving element(s) and a “bearing pair” is formed when a static element and a moving element frictionally bear on one another during the relative motion of blood pump operation. A frictional bearing may comprise a sliding frictional movement where the surfaces of the bearing elements slide over each other or it may be a rolling friction where at least one bearing element roll over a second bearing element.

It is also an objective of this invention to disclose both the most desirable geometric properties of bearing pairs of blood pumps and also the principles of selection of bearing materials for said blood pump bearing pairs. Bearing pairs as used in this invention refer to a static element and a moving element that move relative to each other during blood pump operation and contact each other during at least a portion of said operation so as to limit relative motion of the blood pump elements.

The term “bearing pair” as used in this invention shall also mean the selection of the materials of both the static element and the moving element of a bearing pair. It will be appreciated with reference to the disclosure generally that some materials work well as bearing pairs and others are less favorable bearing pairs. For example, a titanium on titanium bearing pair is an example of a poor bearing pair whereas a PEEK on PEEK is generally a good bearing pair. This invention discloses a number of bearing pairs that perform excellently in terms of low friction and low bearing wear during operation. As depicted in FIG. 5, the pump assembly 300 further comprises a restrictor 314 configured for delivery in a collapsed state and expandable at the site of therapy so as to appose the wall of the blood vessel. In the restrictor expanded state, the only pathway for fluid to move from one side of the restrictor to the other is through the pump housing 301. The restrictor 314 further comprises proximal neck 318 and distal neck 319 both of which are welded to the housing 301 so as to form a sealed annular chamber in the body of the restrictor 314.

In an embodiment, the restrictor 314 comprises a compliant balloon 315. The pump assembly 300 comprises inlet openings 307 which are configured to allow blood into the pump housing 301 and outlet openings 308 which are configured to allow blood to exit the pump housing 301. The impeller 302 comprises an impeller core 306 and an impeller blade 309. The impeller core 306 tapers along its length getting progressively larger distally. The impeller core 306 comprises a gently tapering frustrum at its proximal end and it transitions to a parabolically tapering body at its distal end. The impeller 302 comprises two radially opposing impeller blades 309 that extend radially from the impeller core 306.

The impeller blades comprise an impeller blade proximal end 310 and an impeller blade distal end 311. The impeller blades 309 are configured to propel fluid axially within the housing 301 at the impeller proximal end 310 and the impeller blade 309 are configured to propel fluid radially through the outlets 308 at the impeller distal end 311. The catheter shaft 316 comprises a plurality of control lumens 335. The control lumens 335 comprise one or more of restrictor inflation and deflation lumen, pressure sensor lumen, drive shaft lumen and medicant delivery lumen.

The bearing arrangement 321 is configured such that when the impeller is biased proximally a small impeller distal gap 337 exists between the distal bearing surfaces 325 and 327. The bearing arrangement 321 is configured such that when the impeller is biased distally that a small impeller proximal gap 336 exists between the proximal bearing surfaces 324 and 326. In one embodiment the impeller proximal gap 336 and/or impeller distal gap 337 is configured to retain a lubricant material in the gap. In one embodiment the impeller proximal gap 336 and/or impeller distal gap 337 is configured to allow biological fluids into the gap. In one embodiment the impeller proximal gap 336 and/or impeller distal gap 337 is configured such that the shear rate in the gap is sufficiently high to prevent blood plasma from forming a clot. Indeed, the principles described with respect to FIG. 3 can also be applied to the impeller proximal gap 336 and/or impeller distal gap 337 of FIG. 5.

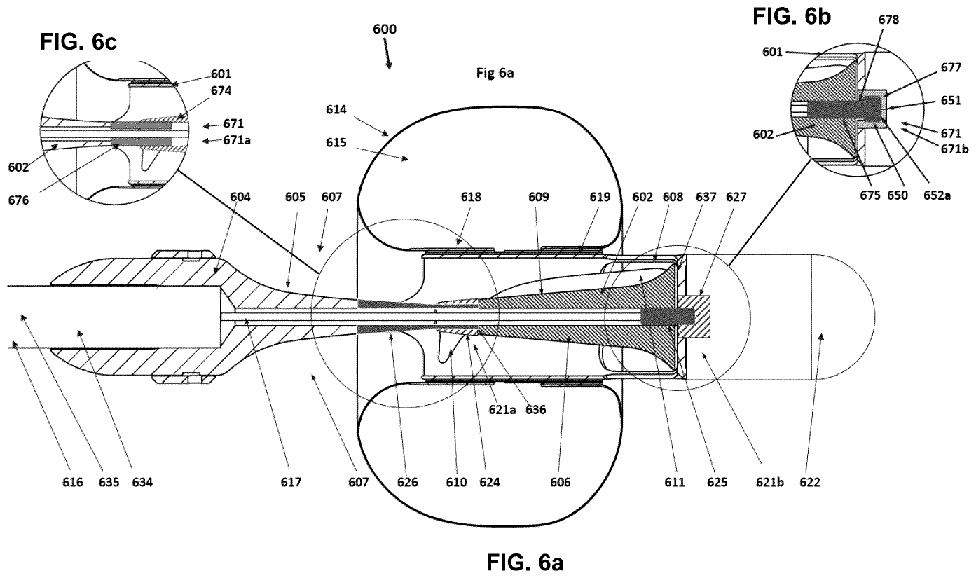

In an alternative embodiment of a pump assembly 600 of intravascular catheter system 1, in accordance with the present invention is described in FIG. 6a. In the embodiment, a pump assembly 600 comprises a pump housing 601, an impeller 602, a catheter shaft 616, a supporting cuff 604, a restrictor 614, a drive shaft 617, a catheter tip 622, and a bearing arrangement 621. The bearing arrangement 621 comprises a proximal bearing arrangement 621a and a distal bearing arrangement 621b. The proximal bearing arrangement 621a comprises a static cuff bearing 626 and an impeller proximal bearing 624. The static cuff bearing 626 is fixedly attached to or integral with a distal region of support cuff 604 and is sized to fit inside the ID of the impeller proximal bearing 624. The static cuff bearing 626 is configured to bear rotationally on the ID of the impeller proximal bearing 624 and comprises a high modulus hard wearing material with a tapered region as it exits the ID of the impeller proximal bearing 624. The static cuff bearing 626 is configured to prevent the impeller 602 from moving axially proximally in operation. In one embodiment the static cuff bearing 626 comprises an abutment surface said abutment surface comprising a bearing abutment surface and configured to bear on a bearing surface of the impeller 602, the bearing surface of the impeller 602 internal to the impeller core 606. In one variation the bearing surface of the impeller 602 comprises a stepped annular surface internal to the impeller core 606. In one variation the static cuff bearing 626 comprises a bearing surface with an angular component that acts to centre the impeller 602 relative to the axis of the pump housing 602 as well as an angular component that acts to abut proximal movement of the impeller 602. In one embodiment a tapered region of the static cuff bearing 626 is configured to act simultaneously as a cylindrical bearing surface and a conical bearing surface or a stepped annular bearing surface at the same time.

The distal bearing arrangement 621b comprises an impeller distal bearing 625 and a distal static bearing 627. The impeller distal bearing 625 is fixed to the impeller distal region and its central axis is configured to coincide with the central axis of the impeller 602. The impeller distal bearing 625 comprises a distal spigot that extends into a bearing orifice in the distal static bearing 627. The distal static bearing 627 comprises two bearing surfaces with the distal spigot of the impeller distal bearing 625. The first of said two bearing surfaces is a cylindrical surface and the second is a disc shaped distal surface. In one variation the disc shaped bearing surface comprises an annular bearing surface of the distal static bearing 627.

In an embodiment, the cylindrical bearing surface and the annular or disc shaped bearing surface comprise a continuous surface. In another variant, the cylindrical bearing surface and the annular or disc shaped bearing surface comprise a non-continuous surface.

The catheter shaft 616 is connected to the supporting cuff 604 and extends proximally and exterior of the patient and facilitates positional control and operational control by the physician of the catheter system 1.

The static cuff bearing 626 and impeller proximal bearing 624, and the impeller distal bearing 625 and distal static bearing 627 constitute bearing pairs as previously described.

In one embodiment a first part of the impeller distal bearing 625 and distal static bearing 627 bearing pair may comprise a homopolymer and the second a composite, the composite may further comprise the homopolymer of the first part as at least one constituent. In one variation the homopolymer comprises a PEEK, nylon, polyimide or polyacetal composite and the composite further comprising one or more of PTFE, Graphite, and Carbon fiber as composite fillers. In another embodiment the arrangement of the bearing pair may be in an opposite configuration and still maintain favorable performance. Similar variations may also be embodied in the static cuff bearing 626 and impeller proximal bearing 624 bearing pair as could any suitable bearing pair comprised of the materials listed in Table 1.

The catheter shaft 616 comprises a straight configuration wherein the axis of the catheter shaft 616 comprises a straight line and a curved configuration wherein the central axis of the catheter shaft 616 comprises a curved undulating line that is determined by the catheter shaft 616 conforming to the anatomy of the patient. In an embodiment, the catheter shaft 616 comprises a flexible polymeric shaft with a reinforcement 634, the reinforcement 634 is configured to allow the catheter shaft 616 to flex and conform to the curved configuration without extending or compressing the neutral axis of the catheter shaft 616. The non-extension and/or non-compression of the neutral axis of the catheter shaft 616 protects the drive shaft 617 from axial compressive or extensive forces and this is desirable as it prevents those forces from being transmitted to the impeller 602 at the distal end or the motor 131 or 631 at the proximal end of the drive shaft 617.

In one variation the catheter shaft 616 with the reinforcement 634 is configured such that when transitioned from the straight configuration to the curved configuration that the length of the neutral axis of the catheter shaft 616 extends by less than 0.4%. Preferably the catheter shaft 616 with the reinforcement 634 is configured such that when transitioned from the straight configuration to the curved configuration that the length of the neutral axis of the catheter shaft 616 extends by less than 0.2%. Preferably the catheter shaft 616 with the reinforcement 634 is configured such that when transitioned from the straight configuration to the curved configuration that the length of the neutral axis of the catheter shaft 616 extends by less than 0.1%.

In one variation the catheter shaft 616 with the reinforcement 634 is configured such that when transitioned from the straight configuration to the curved configuration that the length of the neutral axis of the catheter shaft 616 compresses by less than 0.4%.

Preferably the catheter shaft 616 with the reinforcement 634 is configured such that when transitioned from the straight configuration to the curved configuration that the length of the neutral axis of the catheter shaft 616 compresses by less than 0.2%. Preferably the catheter shaft 616 with the reinforcement 634 is configured such that when transitioned from the straight configuration to the curved configuration that the length of the neutral axis of the catheter shaft 616 compresses by less than 0.1%.

In some embodiments, the catheter shaft 616 comprises a plurality of control lumens 635. The control lumens 635 may comprise one or more of restrictor inflation and deflation lumen, pressure sensor lumen, drive shaft lumen and medicant delivery lumen. The catheter shaft 616 further comprises a multilumen tubing. A first central lumen of the catheter shaft 616 is configured to house the drive shaft 617 and allow it to operate at high rotational speeds of between 10,000 and 60,000 RPMs without generating heat or friction. At least one second lumen of the catheter shaft 616 comprises a peripheral lumen (i.e., it's not on the central axis of the catheter 616) and is configured to sense pressure in the region of therapy or adjacent (proximal or distal) of the region of therapy. At least one pressure sensing lumen may comprise a lumen configured to hold a pressure sensor. At least one pressure sensing lumen may comprise a lumen configured to transmit pressure from a region of therapy or adjacent a region of therapy to a pressure sensor that is remote or spaced apart from the region of therapy. At least one pressure sensing lumen comprises an opening or a port that establishes fluidic channel between the region of therapy or adjacent a region of therapy and the pressure sensing lumen. At least one third lumen of the catheter shaft 616 comprises an inflation lumen and this lumen is configured to allow a user to inflate the restrictor balloon 615 from exterior of the patient. This inflation lumen comprises a luer fitting at its proximal end and provides a sealed channel for fluid to flow from the proximal luer through the at least one third lumen of the catheter shaft 616 and the at least one third lumen extends from the catheter shaft 616 into the restrictor 614 until an opening at the distal end of the at least one third lumen is fluidically connected with the reception space in the interior of the restrictor 614. At least one fourth lumen of the catheter shaft 616 comprises a flushing lumen. The flushing lumen of the catheter shaft 616 comprises a peripheral lumen and extends from a flushing port exterior of the patient through the catheter shaft 616 and allows the user to deliver a water based flush solution to the proximal and/or distal gaps that exist between the impeller 602 and the cuff static bearing 622 (proximally) and the impeller distal gap 637 (distally). The bearing arrangement 621 may be configured such that when the impeller is biased distally that a small impeller proximal gap 636 exists between the proximal bearing surfaces 624 and 626. In one embodiment the impeller proximal gap 636 and/or impeller distal gap 637 is configured to retain a lubricant material in the gap. In one embodiment the impeller proximal gap 636 and/or impeller distal gap 637 is configured to allow biological fluids into the gap. In one embodiment the impeller proximal gap 636 and/or impeller distal gap 637 is configured such that the shear rate in the gap is sufficiently high to prevent blood plasma from forming a clot.

The flush solution is selected from the group comprising of sodium chloride solution, a glucose solution, a dextrose solution, a heparin solution, a sodium bicarbonate solution, a phosphate buffered solution, and a mixture thereof.

The supporting cuff 604 is fixed to the pump housing 601 in a manner that connects and aligns the pump housing 601 and the supporting cuff 604. The supporting cuff 604 is configured such that its distal end is concentric with the central axis of the pump housing 601. The supporting cuff 604 in turn aligns and maintains alignment between the proximal end of the impeller 602 and the central axis of the housing 601. In some embodiments, the impeller 602 comprises an impeller core 606 and an impeller blade 609. The impeller blades may comprise an impeller blade proximal end 610 and an impeller blade distal end 611. The impeller core 606 tapers along its length getting progressively larger distally. The impeller core 606 comprises a gently tapering frustrum at its proximal end and it transitions to a parabolically tapering body at its distal end. The impeller 602 comprises two radially opposing impeller blades 609 that extend radially from the impeller core 606.

In an embodiment, the distal end of the supporting cuff 604 comprises an abutment surface that engages with a stepped abutment surface of the impeller 602 and the cuff abutment surface supports in maintaining the axial position impeller 602. The supporting cuff 604 comprises a cuff transition region 605 which is configured to direct blood into the housing 601 without fluid turbulence or recirculation. The pump housing 601 extends from the enlarged proximal section of the supporting cuff 604 to the catheter tip 622 and comprises at least one fluid inlet 607, at least one fluid outlet 608 and a pump housing body between the inlets 607 and the outlets 608. The restrictor 614 is mounted on the pump housing body region between the fluid inlets 607 and the fluid outlets 608. In some embodiments, the restrictor 614 further comprises proximal neck 618 and distal neck 619 both of which are welded to the housing 601 so as to form a sealed annular chamber in the body of the restrictor 614. The pump housing 601 is sealingly connected to the catheter tip 622 at its distal end. The inner diameter of the pump housing 601 comprises a high precision cylindrical bore over at least a portion of the length of the housing 601.

The drive shaft 617 comprises a rigid elastic or super elastic material and is configured to have a low second moment of area (denoted ‘I’). The formula for second moment of area for a rod like drive shaft 617 is outlined in Equation 7 below.

$$I = ( π {r}^{4} ) / 4 \qquad Equation 7$$

A drive shaft 617 that combines a low second moment of area in combination with a rigid drive shaft material allows the drive shaft 617 to effectively transmit rotational energy from the motor to the impeller 602 without excessive friction and while allowing the catheter shaft 617 to conform to the anatomy of the patient. In an embodiment, the drive shaft 617 comprises an elongate rod like member. In another embodiment, the drive shaft 617 comprises an elongate rod like composite structure. In one embodiment the second moment of area of the drive shaft 617 is less than 4×10⁻⁴ mm⁴. Preferably, the second moment of area of the drive shaft 617 is less than 2×10⁻⁴ mm⁴. More preferably, the second moment of area of the drive shaft 617 is less than 8×10⁻⁵ mm⁴. In one embodiment the drive shaft 617 comprises a high aspect ratio drive shaft 617. As used in this invention the aspect ratio of the drive shaft comprises the ratio of the diameter of the drive shaft 617 to the length of the drive shaft 617 (wherein the length comprises the distance from the proximal end of the impeller to the coupling of the drive shaft 617 with the motor). The reinforced catheter shafts 616 of this invention allow for high aspect ratio drive shafts 617 because the reinforced catheter shafts 616 are configured to protect the drive shafts 617 from extending or compressing forces when the drive shafts 617 are moved from the straight configuration to a curved (in use) configuration. Furthermore, the reinforced catheter shafts 616 of this invention protect the drive shafts 617 from excessive bending forces in use. These features of the reinforcement allow drive shafts 617 with a high aspect ratio to be designed and used effectively. In one embodiment the drive shaft 617 comprises an aspect ratio of 1:750. In one embodiment the drive shaft 617 comprises an aspect ratio of 1:1000. In one embodiment the drive shaft 617 comprises an aspect ratio of 1:1500. In one embodiment the drive shaft 617 comprises an aspect ratio of 1:1750.

As depicted in FIGS. 6b and 6c, the bearing arrangement 671 comprises a proximal bearing arrangement 671a and a distal bearing arrangement 671b. In this alternative embodiment of the bearing arrangement 671, maintaining the axial position of the impeller 602 is achieved entirely by the distal bearing arrangement 627b. The distal bearing arrangement 627b comprises a rotating impeller distal bearing 675 and a static distal bearing 677. These pair of bearing elements provide axial stability in both axial directions as well as maintaining the impeller 602 concentric with the axis of the pump housing 601.

The impeller distal bearing 675 is integral with the impeller 602 or is fixedly attached to the impeller 602, is coaxial with the impeller 602 and comprises at least partially a cylindrical shaft that extends from the distal surface of the impeller 602.

In an embodiment, the impeller distal bearing 675 comprises a bearing head 650. The bearing head 650 further comprises a distal surface 652a (non-bearing), a proximal bearing surface 652b and a cylindrical body bearing surface 652c between the proximal and distal bearing surfaces.

The static distal bearing 677 comprises a female bearing surface. The female bearing surface comprises a cylindrical hole. The cylindrical hole comprising an annular grove the hole and groove configured to accommodate the head 650 of the impeller distal bearing 675.

The bearing head 650 is dimensioned such that the length of its head 650 is slightly shorter than the annular groove in the static distal bearing 677 into which it fits. This means that there is a small gap between the two and this gap allows a small amount of axial movement of the impeller 602, typically 25-50 micrometers. Since, in operation, the impeller 602 is biased proximally the proximal bearing surface 652b of the head 650 bears on the static distal bearing 677. This means that the distal surface 652a is spaced apart from the static distal bearing 677 by 25-50 micrometers and as such is not a bearing surface from a geometric factor (GF) perspective.

In an embodiment, some, all, or just parts of the proximal bearing surface 652b and/or the cylindrical body bearing surface 652c are configured to bear against the surfaces of the static distal bearing 677.

In an embodiment, the bearing head 650 of the impeller distal bearing 675 comprises enlarged distal end 650 connected to the impeller distal bearing 675 by a distal bearing neck 678.

In an embodiment, the distal bearing neck 676 is not a bearing surface and comprises a gap between its OD and the ID of the distal static bearing 677.

In an embodiment, the distal bearing neck 678 comprises a bearing surface and is configured to bear against a surface of the distal static bearing 677.

In an embodiment, the static distal bearing 677 comprises a two-part split structure with a split line 651. The split structure allows the impeller distal bearing 675 with an enlarged distal end 650 to be placed inside the static distal bearing 677 without damage or deformation to either part. The split static distal bearing 677 is clamped shut, housing the bearing head 650 of the impeller distal bearing 675 inside. The split static distal bearing 677 is housed in the distal bearing arrangement 671b inside either the distal section of the pump housing 601 or the catheter tip 622. In either scenario, the distal bearing arrangement 671b is configured such that the central axis of the cylindrical hole with annular grove of the static distal bearing 677 is concentric with the axis of the pump housing 601.

In an embodiment, the enlarged distal end 650 comprises a head.

In an embodiment, the enlarged distal end 650 comprises a proximal facing bearing surface, a cylindrical bearing surface and a distal facing bearing surface. Preferably the enlarged distal end 650 and the distal static bearing 627 are configured to minimize the geometric drag factor of its bearing surfaces.

Referring to FIG. 6c, an alternative variant of proximal bearing arrangement 671a of the bearing arrangement 671, in accordance with the present invention is described. The proximal bearing arrangement 671a comprises an impeller proximal bearing 674 and a cuff static bearing 676. The proximal bearing arrangement 671a is configured to maintain the impeller 602 proximal end coaxial with the central axis of the pump housing 601 during the operation.

The proximal bearing arrangement 671a as depicted in FIG. 6c differs from the proximal bearing arrangement 671 as illustrated in FIG. 6a as it has no bearing surface to limit axial movement. This is not necessary with the bearing arrangement 671 because the distal bearing arrangement 671b limits the axial movement of the impeller 602 in both axial directions. The impeller proximal bearing 674 comprises a geometrically profiled outer (external) surface configured to ensure non-turbulent fluid flow in operation and an inner lumen configured to receive and bear against the distal part of the cuff static bearing 676.

The impeller proximal bearing 674 comprises a biocompatible material, with a smooth surface finish (RA value less than 150 nanometers) and further comprises a wear resistant inner surface. The cuff static bearing 676 comprises the distal region of the supporting cuff 604 and comprises a cylindrical body.

In an embodiment, the cylindrical body comprises a guide pin that is configured for insertion into the impeller proximal bearing 674 inner lumen. The guide pin of the cuff static bearing 676 comprises a bearing surface with at least a portion of the impeller proximal bearing 674 inner lumen. The cuff static bearing 676 also comprises an inner lumen that is configured to receive the drive shaft 617. The inner lumen of cuff static bearing 676 is sized to provide a clearance fit for the drive shaft 617.

It will be appreciated that the configurations shown in FIGS. 5 and 6a, 6b, and 6c utilize bearing surfaces with a reduced bearing surface area compared to other figures such as that shown in FIG. 3a. Configurations with smaller bearing surface contact areas benefit from bearing pairs comprising materials with exceptional wear resistance. In one such embodiment desirable bearing pair surfaces would be comprised of DLC to DLC, DLC to a Conundrum or Ceramic, or a Conundrum or Ceramic to a Conundrum or Ceramic. However, depending on the lifecycle of the device in question previously disclosed variations may also suffice.

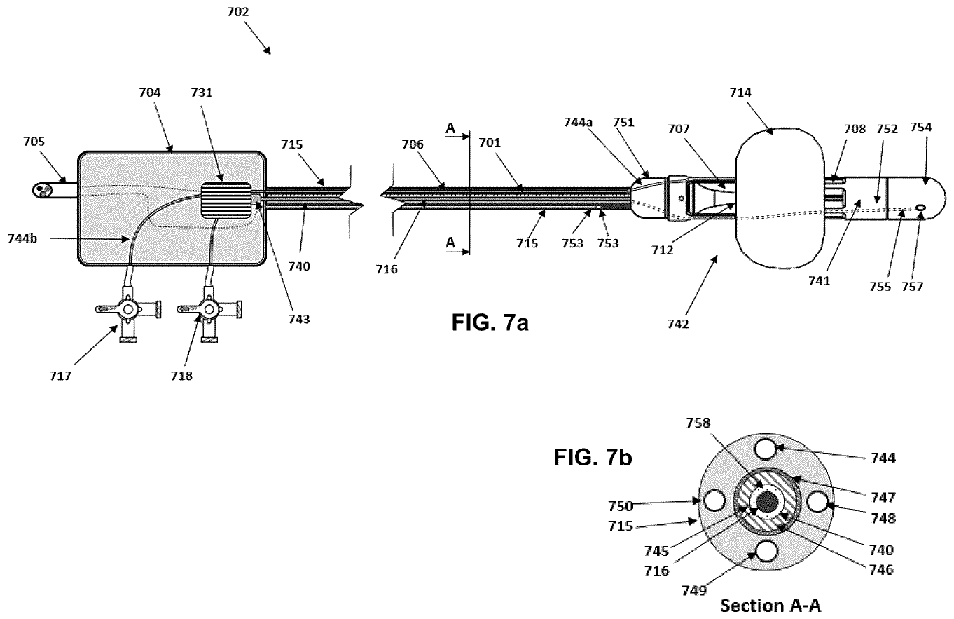

Referring to FIG. 7a and FIG. 7b, an alternative embodiment of a blood pump 702, in accordance with the present invention is described. The blood pump 702 is compatible with the sheaths and consoles illustrated in FIGS. 2a-2b. It will be understood that the blood pump 702 can be integrated with the sheath 3, console 7 and other elements of the catheter system 1.

The blood pump 702 comprises a pump assembly 742, a catheter shaft 715, a drive shaft 716 and a manifold 704. The pump assembly 742 comprises a pump housing 741, impeller 712, catheter tip 754, bearing arrangement 752, supporting cuff 751, and restrictor 714. The pump assembly 742 further comprises the distal segment of the inflation tube 744 which extends from the catheter shaft 715 into the restrictor 714. The catheter shaft 715 comprises an elongate structure that is connected to the pump assembly 742 at its distal end and the manifold 704 at its proximal end.

The catheter shaft 715 is configured for partial insertion into a mammalian body, its distal end configured for advancement through the lumen of sheath 3 to a site of therapy and its proximal end configured so as to allow operation of the blood pump 702 by the operator external of the patient.

In an embodiment, the blood pump 702 comprises a pump assembly 742, a catheter shaft 715, a drive shaft 716 and a motor 731. The pump assembly 742 is comprised of a pump housing 741, an impeller 712, a restrictor 714, a pump inlet 707 and a pump outlet 708. The restrictor 714 is downstream of the pump inlet 707 and the pump outlet 708 lies downstream of the restrictor 714. The pump assembly is configured in use to pump bodily fluid from the vascular network upstream of said restrictor 714 to the vascular network down stream of said restrictor 714. The drive shaft 716 is coaxially connected to the impeller 712 at its distal end and is coaxially coupled to the rotor shaft of the motor 731 at its proximal end and the drive shaft 716 is configured to transmit motor torque or motor rotational energy from the motor 731 to the impeller 712.

In an embodiment, the catheter shaft 715 comprises a central lumen 745, the central lumen 745 being configured to receive the drive shaft 716 and facilitates the rotational operation of the drive shaft 716. The central lumen 745 is preferably sized relative to the drive shaft 716 so as to define an annular gap between the drive shaft 716 and the central lumen 745, the annular gap being at least partially filled with an annulus of viscous grease 740. The annulus of viscous grease 740 surrounds the drive shaft 716 and extends along at least part of the length of the central lumen 745.

In an embodiment, the annulus of viscous grease 740 comprises an aspect ratio (annulus length/annulus thickness) and the aspect ratio of the annulus of viscous grease 740 is greater than 5,000. Preferably, the aspect ratio of the annulus of viscous grease 740 is greater than 10,000. Preferably, the aspect ratio of the annulus of viscous grease 740 is greater than 15,000. More preferably, the aspect ratio of the annulus of viscous grease 740 is greater than 20,000. More preferably, the aspect ratio of the annulus of viscous grease 740 is greater than 25,000. Even more preferably, the aspect ratio of the annulus of viscous grease 740 is greater than 30,000.

The viscous grease may be comprised of one of more components.

In an embodiment, the grease is comprised of a base oil and a thickener. The base is preferably Poly-alpha-olefin with a polyurea thickener. The base oil viscosity and quantity of thickener can be varied to accommodate pressure head and drive shaft liner velocity requirements. Alternatively, thickeners can be utilized to improve the water resistance of the grease such as aluminum, lithium, or calcium complexes.

In another embodiment, a Poly-alpha-olefin base oil is paired with an aluminum complex thickener to improve water washout and grease migration. In another embodiment, Perfluoropolyether (PFPE) based oils with PTFE thickeners, PFPE-/PTFE-pastes, and high and medium consistency dimethyl silicone greases may be utilized to form the grease barrier.

In an embodiment, the annulus of viscous grease 740 is bound in place by the inner surface of the drive shaft liner 746. The catheter shaft 715 may comprise a plurality of peripheral lumens 706 the plurality of peripheral lumens configured to allow the operation of the catheter system 1. One or more of said plurality of lumens may comprise an inflation lumen. One or more of said plurality of lumens may comprise a pressure sensing lumen. One or more of said plurality of lumens may comprise a flush lumen.

In an embodiment, the viscous grease 758 comprises self-cohesive fluid when placed in biological fluid environment. The viscous grease 758 may comprise a saline contact angle that is greater than 60°. The viscous grease 758 may comprise a saline contact angle that is greater than 70°. The viscous grease 758 may comprise a saline contact angle that is greater than 80°. The viscous grease 758 may comprise a saline contact angle that is greater than 90°.

In an embodiment, the annulus of viscous grease 740 is configured to prevent biological fluid ingress into the annulus between the drive shaft 716 and the central lumen 745.

In an embodiment, the drive shaft 716 comprises a longitudinal rod and the drive shaft 716 is configured to be resistant to kinking when operating at rotational speeds of between 10,000 and 60,000 RPMs in tortuous body anatomies. Preferably, the drive shaft 716 comprises a highly elastic metallic rod. In another variant, the drive shaft 716 comprises a super elastic or shape memory metal.

In an embodiment, the drive shaft 716 is geometrically configured to operate within its elastic limits when the catheter shaft 715 is placed in tortuous anatomy. In another variant, the drive shaft 716 is geometrically configured to operate within its elastic limit by selecting a drive shaft 716 with a second moment of area of less than 4×10⁻⁴ mm⁴. Preferably, the geometrically configured drive shaft 716 operates within its elastic limit by selecting a drive shaft 716 with a second moment of area of less than 2×10⁻⁴ mm⁴. Preferably, the geometrically configured drive shaft 716 operates within its elastic limit by selecting a drive shaft 716 with a second moment of area of less than 8×10⁻⁵ mm⁴.

FIG. 7b illustrates a cross sectional view of the catheter shaft 715 along the section A-A. The catheter shaft 715 comprises a drive shaft liner 746, annular reinforcement layer 747 and a plurality of control lumens 706. The drive shaft liner 746 comprises an elongate tubular member with a central lumen 745 and is configured to receive the drive shaft 716. The inner diameter of the drive shaft liner 746 is sized to allow the drive shaft 716 to rotate at high speeds, typically 15,000 to 60,000 RPMs without interference. Conversely, the inner diameter of the drive shaft liner 746 is sized to provide the minimum clearance possible between the drive shaft 716 and the inner diameter of the drive shaft liner 746 so as to prevent the ingress of bodily fluids into the gap between the drive shaft liner 746 and the drive shaft 716.

Preferably, the thickness of the annular gap between the drive shaft liner 746 and the drive shaft 716 is 0.01 mm+0.02/−0.005.

The catheter shaft 715 further comprises an annulus of viscous grease 740 surrounding the drive shaft 716 and bound in place by the inner surface of the drive shaft liner 746. The annulus of viscous grease 740 comprises a column of viscous grease 758 the column extending at least a part of the length of the catheter shaft 715. The annular column of viscous grease 740 is configured to provide lubrication to the rotating drive shaft 716 while being resistant to axial migration during the operation.

In an embodiment, the annulus of viscous grease 740 comprises an aspect ratio the aspect ratio being calculated by dividing the length of the annulus by the thickness of the annulus as described in equation 8.

$$Grease Annulus Aspect Ratio = \frac{Length of Annulus ( mm )}{Thickness of Annulus ( mm )} \qquad Equation 8$$

Higher aspect ratios are more conducive to maintaining the viscous body of fluid in the annulus and preventing the ingress of bodily fluids. Preferably, the thickness of the annulus is chosen to ensure that the aspect ratio of the annular column of grease is greater than 500. Preferably, the aspect ratio of the annular column of grease is greater than 1,000. More preferably, the aspect ratio of the annular column of grease is greater than 5,000. More preferably, the aspect ratio of the annular column of grease is greater than 10,000. Even more preferably, the aspect ratio of the annular column of grease is greater than 20,000. Even more preferably, the aspect ratio of the annular column of grease is greater than 25,000. Even more preferably, the aspect ratio of the annular column of grease is greater than 30,000.

The resistance of the annulus of viscous grease 740 to axial migration makes it difficult for bodily fluids to ingress into the annular gap and this in turn prevents bodily fluids migrating proximally through the lumen of the drive shaft liner 746 to the motor 731.

The drive shaft 716 is configured to induce and even field of circumferential shear forces on the annular column of grease 740 without compressing the viscous grease 758 of the column. The absence of compression of the viscous grease 758 in operation prevents the viscous grease 758 from migrating which in turn prevents bodily fluid ingress. The viscous grease 758 is selected to be viscous and self-cohesive in biological fluid setting. Self-cohesive greases are viscous greases 758 wherein the molecular forces of attraction between adjacent grease molecules are greater that the forces of attraction between a grease molecule and molecules of biological fluid that are adjacent the grease molecule at the grease/biological fluid boundary. When a viscous grease is self-cohesive, it is more difficult for biological fluid to ingress a gap occupied by the grease.

A simple biological fluid is normal saline and is typically a solution of 0.90% w/v of sodium chloride (NaCl). The ability of a viscous grease to repulse bodily fluids can be measured by measuring the degree to which the grease repels normal saline. This can be achieved with a simple contact angle test where a droplet (5 microliters) of normal saline is dropped onto a flat surface coated with the viscous grease and the contact angle between the grease and the droplet of normal saline is measured. The contact angle is defined as the angle formed by a liquid (normal saline) at the three-phase boundary where a liquid (saline), gas (air), and solid (viscous grease) intersect. In respect to the present invention, the viscous grease is not a solid surface but when applied to a solid surface for the test it behaves as a solid for the purposes of measuring the interaction between the biological fluid and the viscous grease.

Preferably, viscous greases 758 of the present invention have a saline contact angle of greater than 50°. Preferably, viscous greases 758 have a saline contact angle of greater than 60°. Preferably, viscous greases 758 have a saline contact angle of greater than 70°. Preferably, viscous greases 758 have a saline contact angle of greater than 80°. Preferably, viscous greases 758 have a saline contact angle of greater than 90°.

In an embodiment, the drive shaft 716 comprises an elongate cylindrical member with a smooth continuous surface (without breaks, gaps or joints), the smooth continuous surface of the drive shaft 716 circumferentially shearing the grease in operation without compressing the grease or otherwise inducing axial flow in the grease.

In an embodiment, the drive shaft comprises a rodlike elongate cylindrical member.

In an embodiment, the drive shaft 716 comprises an outer skin, the outer skin comprising a continuous, smooth, and unbroken surface.

In an embodiment, the drive shaft 716 comprises a hypodermic tube, the hypodermic tube comprising a continuous, smooth, and unbroken surface.

In an embodiment, the drive shaft 716 comprises a nitinol rod, a nitinol wire or a nitinol hypodermic tube wherein the outer surface of the nitinol rod, a nitinol wire or a nitinol hypodermic tube comprises a continuous, smooth and unbroken surface. Preferably, the diameter of the drive shaft 716 is as small as possible. A smaller drive shaft diameter means that the drive shaft 716 induces less shearing of the grease and so less breakdown of the grease during long term operation of the blood pump 702.

In an embodiment, the diameter of the drive shaft 617 is less than 0.031 mm.

In an embodiment, the diameter of the drive shaft 617 is less than 0.026 mm.

In an embodiment, the diameter of the drive shaft 617 is less than 0.021 mm.

In an embodiment, the drive shaft liner 746 comprises a tough, wear resistant and low friction material. The drive shaft liner 746 is made of material comprising a homopolymer or a blended material. Suitable homopolymer or blended material comprise one or more of the following: (i) PTFE, (ii) PEEK, (iii) Polyimide, (iv) Polyamide, (v) Polyacetal or (vi) UHMWPE.

In an embodiment, the drive shaft liner 746 is made of material comprising a composite material. Suitable composite material comprises one or more of the homopolymers with one or more of the following filler or reinforcing materials: (a) PTFE, (b) carbon fiber, (c) graphite, (d) glass fiber, (e) polyimide. In this embodiment, the reinforcement agents may comprise up to 50% of the volume of the drive shaft liner 746.

The catheter shaft 715 further comprises a reinforcement layer 747. The reinforcement layer 747 is configured to allow the catheter shaft 715 to flex and conform to the curvature of the anatomy of the patient but the reinforcement prevents gross axial deformation (either negative or positive) ensuring that the length path of the drive shaft 716 is substantially constant irrespective of the tortuosity of the patient anatomy. The role of the drive shaft 716 in maintaining the length path of the drive shaft 716 further prevents the drive shaft 716 from inducing axial flow on the annular column of grease 740.

The catheter shaft 715 comprises a plurality of control lumens 706. The plurality of control lumens comprises a number of axial lumens extending at least part of the length of the catheter shaft 715. The plurality of control lumens 106 are arranged such that their centers are substantially on a pitched circle diameter. A first control lumen of the plurality of control lumens 706 comprises an inflation lumen 744. The inflation lumen 744 extends substantially the entire length of the catheter shaft 716. Its distal end sealingly merges with the balloon inflation tube 744a which extends from the catheter shaft 715 to the restrictor 714. The proximal end of the inflation lumen sealingly merges with the manifold inflation tube 744b which is connected to a first stopcock 717 in the manifold 704.

In an embodiment, a second control lumen of the plurality of control lumens 706 comprises a first sensor lumen 748. The first sensor lumen 748 is configured to house a first pressure sensor 753. The first pressure sensor 753 comprises a sensing element at its distal end and at least one transmission cable extending there from. The first sensor lumen 748 further comprises a port 753 at its distal end and the port 753 is configured to allow bodily fluid to ingress into the first sensor lumen 748 and transmit bodily pressure to the first pressure sensor 753.

In an embodiment, a third control lumen of said plurality of control lumens 706 comprises a second sensor lumen 749. The second sensor lumen 749 comprises a pressure sensing lumen and is configured to house a second pressure sensor 755 and sense pressure on the distal side of the restrictor 714 through a second port 757 on the catheter tip 754. Data taken from the first pressure sensor 753 and second pressure sensor 755 each have clinical value as separate measurements. However, data taken from the first pressure sensor 753 and second pressure sensor 755 can be compared to monitor the pressure drop created by the pump arrangement 742 across the restrictor 714 during the therapy.

In an embodiment, a fourth control lumen of the plurality of control lumens 706 comprises a fluid flush lumen 750. The fluid flush lumen 750 is configured to allow biocompatible fluids like saline and glucose solutions to be flushed through the gaps between the impeller 712 and the adjacent proximal and distal static surfaces.

The catheter shaft 715 is fixedly coupled to the manifold 704 at its proximal end. The manifold 704 comprises an enclosure 758, motor 731, catheter PCB 735, first stopcock 717, second stopcock 718 and connecting cables 705. The enclosure 758 which houses the contents of the manifold 704 comprises a molded plastic compartmentalized housing. The motor 731 is fixedly mounted to the manifold enclosure 758 and is aligned with the catheter shaft 715 to receive the drive shaft 716 in a coaxial arrangement. The motor 731 further comprises a motor to drive shaft coupler 743. The motor to drive shaft coupler 743 is configured to connect the drive shaft 716 to the motor 731 while allowing a small amount of relative axial motion between the two.

The manifold 704 is also configured to protect and defines a pathway for the cables of the first pressure sensor 748 and second pressure sensor 749 from their point of entry at the proximal end of the catheter shaft 715 to their exit point from the manifold 704 at the start of the connecting cables 705.

FIG. 8a illustrates a bearing test assembly 800, in accordance with an embodiment of the present invention. The bearing test assembly 800 comprises a housing 801, a bearing arrangement 810, connecting cuff 804, catheter shaft 815 and drive shaft 816. The bearing arrangement 810 comprises a bearing shaft 803, first static bearing 805, second static bearing 806, and retaining ring 809. The catheter shaft 815 comprises an elongate flexible tubing with a central lumen and preferably one or more peripheral lumens. The housing 801 comprises a housing separator 824 against which the second static bearing 806 abuts.

The bearing arrangement 810 further comprises a first bearing surface 822a, a second bearing surface 822b and a third bearing surface 822c. The bearing test assembly 800 is configured to allow the interactions between blood and the bearing arrangement 810 to be studied.

FIG. 8b schematically illustrates a blood flow model 850 designed to test the bearing arrangement 810. The blood flow model 850 is configured to facilitate the testing of interactions between mammalian blood and a bearing arrangement 810. The blood flow model 850 comprises a peristaltic pump 851, a water bath 852, with heater and pump 853, a blood bag box 854, height adjustable table 864, motor 865, console 857 and flow loop 859. The flow loop 859 comprises a plurality of interconnected cardiopulmonary tubes 862, interconnected with standard connectors 856, and a blood bag 861 with one cardiopulmonary tubing 862 connected to an inlet of the blood bag 861 and a second cardiopulmonary tubing 862 connected to an outlet of the blood bag 861. The cardiopulmonary tubes 862, connectors 856 and blood bag 861 are interconnected to define a flow loop 859. The blood bag 861 is placed in the blood bag box 854 which is kept at body temperature by its two-way recirculating connection to the water bath 852 with heater and pump 853.

The blood bag box 854 sits on a height adjustable table 864, the height adjustable table 864 being configured allow adjustment of the pressure head at the sample insertion junction 860 by raising the height of the blood bag 861 relative to the insertion junction 860. The sample insertion junction 860 comprises a Y connection to the flow loop 859 with a Touhy Borst seal at the proximal end of the insertion junction 860. The motor 865 is preferably mounted on/in a manifold 847 or another mechanically stabilizing platform. The console 857 is configured to monitor the RPMs of the motor 865 as well as the current that is consumed by the motor 865 as a function of time. Preferably, the console 857 is configured to present data on the RPMs versus time and current versus time in a chart form or in a tabular form.

The test sample 800 is inserted into the blood flow loop 859 at the insertion junction 860 and is advanced until the tip of the test sample 800 is in contact with fluid of the flow loop 859. The peristaltic pump 851 is configured to return blood to the elevated blood bag 861 and the head height of the blood bag 861 determines the blood flow at the insertion junction. The peristaltic pump 851 is adjusted until its flow rate is in balance with the flow at the insertion junction 860. Preferably, the test is conducted with fresh anticoagulated mammalian blood (0 to 4 hours old at start of the test). This is the period where the blood is still alive and clotting function is substantially intact.

When the test sample 800 is in place and blood is flowing through the model 850, the motor 865 is turned on and operated by the console 857 at the target speed. During testing, the console 857 monitors the current being delivered to the motor 865. The current being delivered to the motor 865 correlates with the work being done by the motor 865 and in this test model 850 that is primarily the drag forces in the bearing arrangement 810. The test can be operated for multiple hours and doing so provides time for the development of more challenging blood clots in the bearing arrangement 810.

In general, the clots formed early in the test are soft and less sticky whereas clots formed at later time points are denser, fibrin rich and sticky. Bearing arrangements 810 with a high geometric drag factor are more likely to develop clots that are problematic to the function of the bearing arrangement 810 whereas the bearing arrangements 810 with a low geometric drag factor are less likely to develop clots that impede bearing arrangement 810 function when exposed to and operated in blood over extended durations. At the end of the test, the test sample 800 is removed from the model and gently cleaned of loose blood. The bearing arrangement 810 of the test sample 800 is examined and the nature and volume of clot can be assessed by standard techniques. The current plots recorded by the console 857 during the test can be compared to baseline plots.

The baseline current plot may comprise (i) a plot of the bearing arrangement 810 operated at the target speed in an air environment, (ii) a plot of the bearing arrangement 810 operated at the target speed in a water or saline environment, (iii) a plot of the bearing arrangement 810 operated at the target speed in a simulated blood environment, (iv) a plot of the bearing arrangement 810 operated at the target speed in a blood environment where the baseline is established in the first few minutes of operation before the formation of performance limiting clots. The simulated blood environment at (iii) above may comprise the viscosity of blood but without the clotting properties. Glycerol solutions provide a good simulation of mammalian blood in terms of viscosity.

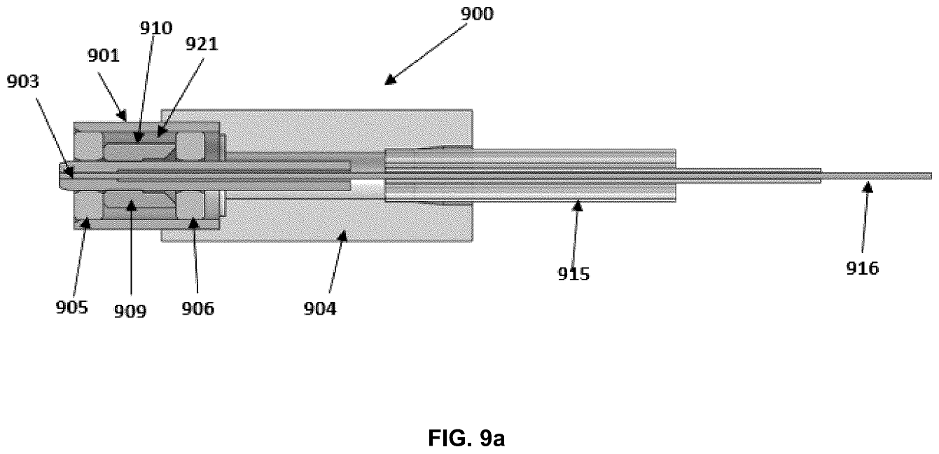

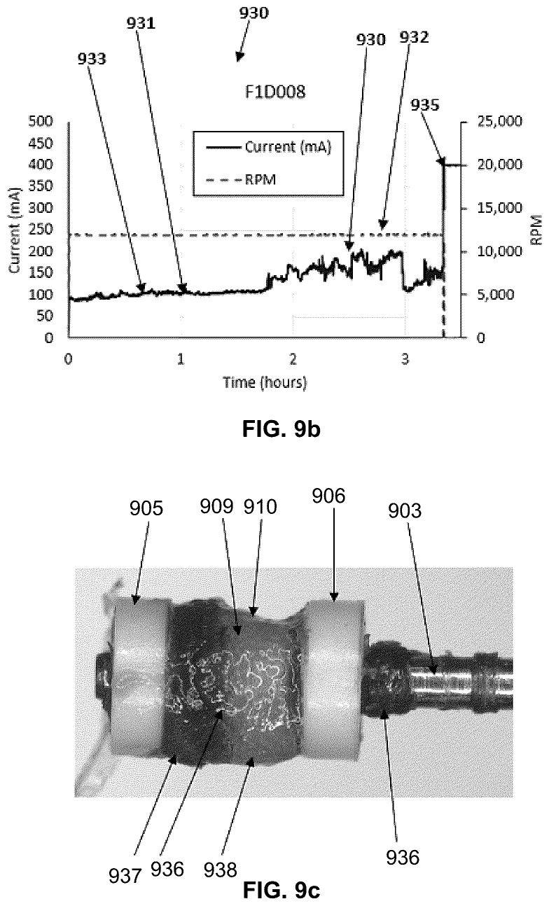

FIG. 9a schematically illustrates a test sample assembly 900 for evaluating a bearing arrangement 910. The test sample assembly 900 is Design F1D008 from Table 2. Design F1D008 has a geometric drag factor of 8.3 mm³ and since this is a relatively high geometric drag factor, it is predictive of the development of clots that may impede the smooth operation of the bearing arrangement 910 over longer time periods. The test sample assembly 900 comprises a bearing arrangement 910, housing 901, connecting cuff 904, catheter shaft 915 and drive shaft 916.

The bearing arrangement 910 comprises a first static bearing 905, a second static bearing 906, a retainer ring 909 and a bearing shaft 903. The retainer ring 909 is coupled to the bearing shaft 903 which in turn is coupled to the drive shaft 916. The retainer ring 909, bearing shaft 903 and the drive shaft 916 rotate together as a sub-assembly when drive shaft 916 is connected to the motor 865 and the motor 865 of the test model 850 is operated. The proximal end of the drive shaft 916 is configured for coaxial coupling to the motor 865 of the test model 850.

The test sample assembly 900 comprises a bearing arrangement 910 that is similar to the bearing arrangement 121 as illustrated in FIG. 2d and so the test sample assembly 900 is a good test model for testing the blood interactions of the bearing arrangement 121 illustrated in FIG. 2d.

FIG. 9b shows a chart 930 of RPMs versus time and motor current versus time for a test sample assembly 900 when evaluated in blood in the test model 850 illustrated in FIG. 8b. The chart 930 shows an RPM plot 932 versus time and a current plot 931 versus time. In this chart, the RPMs are constant throughout the test at 12,000 RPMs. The current plot 931 varies with time and reflects the changing interactions with blood over time. For the first 1 hour and 40 minutes of the test, the current plot 931 comprises a stable current region 933 with the current being very stable at approximately 100 mA. The current response in this period is independent of the geometric drag factor as the blood is still behaving as a viscous fluid. From 1 hour 40 minutes until 3 hours and 15 minutes (where the test ends), the current plot 931 comprises an unstable current response 934 with the current varying between 100 mA and 200 mA and then at 3 hours and 15 minutes the current increases quickly to 400 mA which triggers a stop in the test. This unstable current region 934 comprises a region where blood clots are becoming denser, more organized and more adhesive, and the high geometric drag factor (of 8.3 mm³) of this bearing arrangement 910 is predictive of these more challenging clots will increase the workload on the motor and impede operation of the bearing arrangement 910.

FIG. 9c shows the bearing arrangement 910 removed from the housing 910 after the test in the blood model 850 generated the chart 930. The first static bearing 905, second static bearing 906 and bearing shaft 903 are clearly visible. The retainer ring 909 is covered in clot 936 and indeed this is not surprising as blood can access this region but cannot circulate through the region. It will be noted on closer inspection of the clot 936 that it comprises two parts. The portion of the clot 936 that is adjacent the first static bearing 905 is a red clot 937 while the clot 936 that is adjacent to the second static bearing 906 is white clot 938. Red clot 937 is not adhesive nor is it dense and it indicates that the interface between the retainer ring 909 and the first static bearing 905 was not a bearing surface 122c. This means that in the test the retainer ring 909 was spaced apart from this surface. Conversely, the portion of the clot 936 that is adjacent the second static bearing 906 is a white clot 938. The white clot 938 is denser and more adhesive than the red clot 937 and it is the white clot 938 that is responsible for the drag on the bearing arrangement 910 that shows up in the unstable current region 934. The clot that has formed between the drive shaft 903 and the first static bearing 905 is not visible in this microscopic image but it is likely to also be white clot that is dense and adhesive and forms part of the drag on the bearing arrangement 910. Table 2 predicts that the bearing surfaces between the drive shaft 903 and the two static bearings (905 and 906) contribute 0.79 mm³ to the geometric drag factor whereas the bearing surface between the retainer ring 909 and the second static bearing 906 contributes 7.5 mm³ to the geometric drag factor. The contribution of the latter is almost 10 times the contribution of the former.

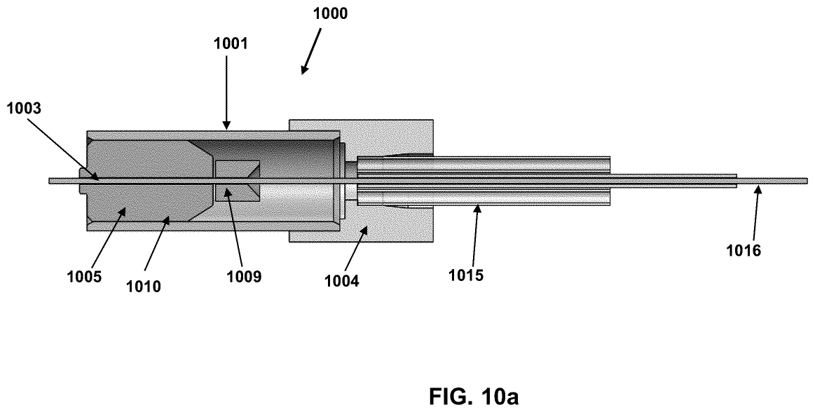

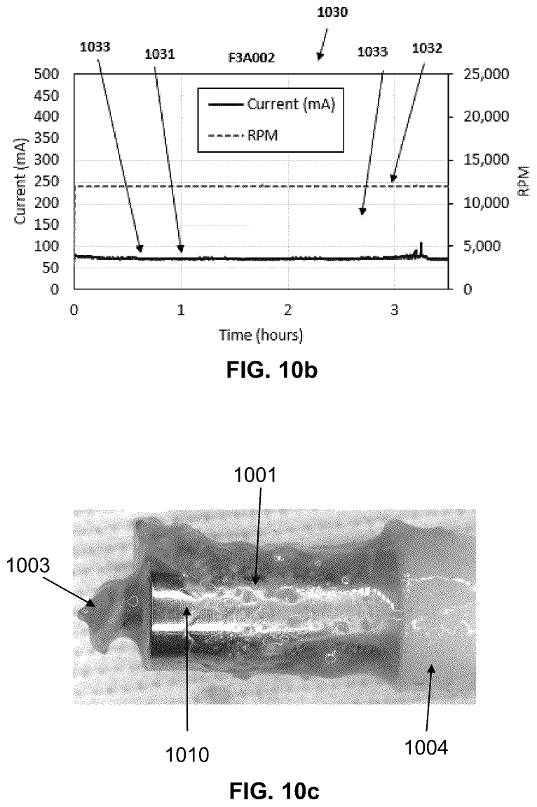

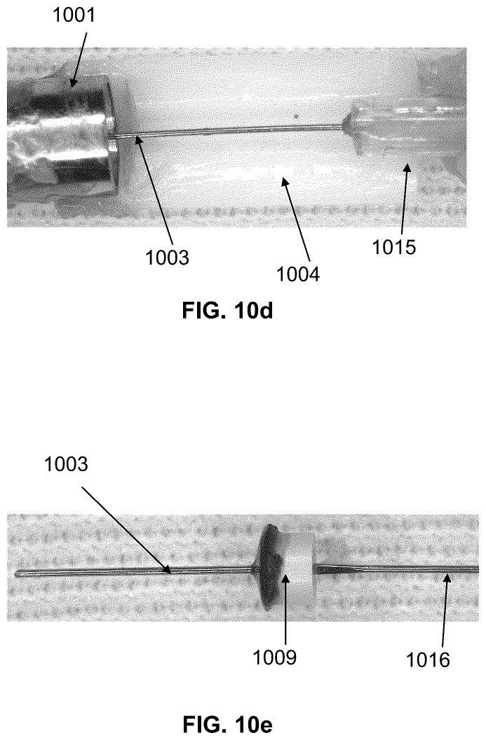

FIG. 10a illustrates an alternative variant of a test sample assembly 1000 suited for use in the test model 850. The test sample assembly 1000 comprises a bearing arrangement 1010 that has the dimensions of the bearing surface of the design referred to as Design F3A002 in Table 3. Design F3A002 has a geometric drag factor of 0.78 mm³ and this GF is approximately 10% of the GF for FID008 above which has a GF=8.3 mm³. This relatively lower geometric drag factor of F3A002 is predictive of a bearing arrangement that does not develop clots that might impede the smooth operation of the bearing arrangement 1010 even over long time periods in vivo. The test sample assembly 1000 comprises a bearing arrangement 1010, housing 1001, connecting cuff 1004, catheter shaft 1015 and drive shaft 1016.

In an embodiment, the bearing arrangement 1010 comprises a bearing block 1005, a retainer ring 1009 and a bearing shaft 1003. The retainer ring 1009 is coupled to the bearing shaft 1003 which in turn is coupled to the drive shaft 1016. The retainer ring 1009, bearing shaft 1003 and the drive shaft 1016 are configured to rotate together as a sub-assembly when drive shaft 1016 is connected to the motor 865 and the motor 865 of the test model 850 is operated. The proximal end of the drive shaft 1016 is also configured for coaxial coupling to the motor 865 of the test model 850.

The test sample assembly 1000 comprises a bearing arrangement 1010 that is similar to the bearing arrangement 221 illustrated in FIG. 3a and so the test sample assembly 900 is a good test model for testing the blood interactions of the bearing arrangement 221 as illustrated in FIG. 3a.

FIG. 10b shows a chart 1030 of RPMs versus time and motor current versus time for a test sample assembly 1000 when evaluated in blood in the test model 850 illustrated in FIG. 8b. The chart 1030 shows an RPM plot 1032 versus time and a current plot 1031 versus time. In this chart 1030, the RPMs are constant throughout the test at 12,000 RPMs. The current plot 1031 varies with time and reflects the changing interactions with blood over time. However, unlike the current plot 931 shown in FIG. 9b, the current plot 1031 of FIG. 10b is stable from time zero until time 3 hours and 30 minutes when the test ends. This current plot 1031 indicates that Design F3A002 has a low propensity to generate or interact with clots that would impede the operation of the bearing arrangement 1010 and so it is a superior bearing arrangement design to Design FID008. This is indeed reflected in its much lower geometric drag factor of 0.78 mm³. The geometric drag factor for the bearing arrangement F3A002 is 1/10^{th} the geometric drag factor forbearing arrangement F1D008. It will also be noted that the baseline current plot for this design (F3A00) is generally lower than the baseline current for design F1D008 OF chart 930 with the current generally in the region of 75 mA throughout the test.

Referring to FIGS. 10c, 10d and 10e, illustrates series of microscopy images of the bearing arrangement 1010 after the test. FIG. 10c shows the test sample before the bearing arrangement 1010 is disassembled. FIG. 10d shows the proximal end of the bearing arrangement 1010 with the bearing shaft 1003 extending from the proximal end of the housing. While some clot has formed it appears to be less dense than in FIG. 9c and does not interfere with the smooth operation of the bearing arrangement 1010. FIG. 10e shows the retainer ring 1009 and the bearing shaft 1003 after disassembly of the bearing arrangement 1010. There is some clot around the perimeter of the retainer ring 1009 but again is does not interfere with the smooth operation of the bearing arrangement 1010.

The exemplary embodiments outlined in FIGS. 9 and 10 highlight the predictive power of the geometric drag factor in predicting the development of clot that might impede the smooth operation of a sliding bearing arrangement of the present invention. The exemplary embodiments incorporated into the test sample assemblies shown in FIGS. 9 &10 can be configured to evaluate any bearing design or bearing pair combination and is not limited to the exemplary designs or bearing pairs disclosed herein.

Referring again to the bearing arrangement 121 illustrated in FIG. 2d, it will be appreciated that the geometric drag factor depends greatly on the dimensions of the bearing surfaces of (i) proximal static bearing 105, (ii) the distal static bearing 106 and (iii) the retainer ring 109. Table 2 presents a table that shows the impact of varying the dimensions of these bearing surfaces on the geometric drag factor for the bearing arrangement 121. It can be seen from the table that the geometric drag factor varied between 1.106 for the F1D001 design to 8.288 for the F1D008 design. In Table 2 the GF for each of the eight designs comprised the sum of the GF contributions of the interactions between: (1) the first static bearing 105 and the drive shaft 116, (2) the second static bearing 106 and the drive shaft 116, and (3) the proximal face of the retainer ring 109 and the first static bearing 105. A GF contribution for the distal surface of the retainer ring 109 did not form part of the calculation because in operation the impeller 102 will bias the retainer ring 109 proximally and so the gap on the distal side of the retainer ring 109 will be greater than 0.2 mm. With design F1D001 to F1D008, the dimensions of the annular bearing surface of the retainer ring 109 has, in particular, a big impact on the overall geometric drag factor for this bearing arrangement 121. It will be appreciated that the dimensions of the elements of the bearing arrangement 121 can be greater or smaller than those illustrated in Table 2, and it is assumed that one of skill in the art would be able to create more extensive tables based on the teachings in this invention.

TABLE 2
Bearing Designs of the Type Show in FIG. 2d

| | | First | Second | | | | | |
|---|---|---|---|---|---|---|---|---|
| | Drive | Static | Static | Retainer | Retainer | | | |
| | Shaft | Bearing | Bearing | Ring | Ring | GF | GF | GF |
| Design | Radius | Length | Length | ID | OD | Cylinders | Annulus | Total |
| Name | (mm) | (mm) | (mm) | (mm) | (mm) | (mm³) | (mm³) | (mm³) |
| Relevant | | | | | | Equation | Equation | |
| Equation | | | | | | 1 | 2 | |
| F1D001 | 0.100 | 0.200 | 0.200 | 0.210 | 0.810 | 0.013 | 1.094 | 1.106 |
| F1D002 | 0.100 | 0.500 | 0.500 | 0.210 | 0.810 | 0.031 | 1.094 | 1.125 |
| F1D003 | 0.250 | 0.200 | 0.200 | 0.510 | 1.110 | 0.079 | 2.587 | 2.665 |
| F1D004 | 0.250 | 0.500 | 0.500 | 0.510 | 1.110 | 0.196 | 2.587 | 2.783 |
| F1D005 | 0.375 | 0.200 | 0.200 | 0.760 | 1.360 | 0.177 | 4.349 | 4.526 |
| F1D006 | 0.375 | 0.500 | 0.500 | 0.760 | 1.360 | 0.442 | 4.349 | 4.791 |
| F1D007 | 0.500 | 0.200 | 0.200 | 1.100 | 1.700 | 0.314 | 7.502 | 7.816 |
| F1D008 | 0.500 | 0.500 | 0.500 | 1.100 | 1.700 | 0.785 | 7.502 | 8.288 |

The best design (F1D001), from a GF perspective, of the eight designs illustrated in Table 2 is the design whereby the outer diameter of the retainer ring 109 is the smallest, where the outer diameter of the drive shaft 116 is the smallest and where the length of the static bearings (105 & 106) is the shortest. It will be obvious that this design can be improved further with respect to GF by further reducing these dimensions. However, material limits will dictate how far this can be pushed because a reduction in the area of the bearing surfaces can, with some materials, increase the wear of those surfaces unless bearing surface material selection and bearing surface pairing dictates that the reduced bearing surface area will resist wearing. Material selection and pairing is covered elsewhere in this disclosure. When calculating the GF for any bearing arrangement of the present invention, it may be necessary to exclude portions of bearing surfaces that possess beveled corners. If parts of the beveled corners result in a bearing gap dimension that are greater than 0.2 mm then these parts of the surface should be excluded from the GF calculation.

Table 3 presents the dimensional parameters and GF calculated values for eight designs with the geometric characteristics of the design of FIG. 3a. The geometric drag factor (GF) for the designs F3A001 to F3A008 depend on the bearing surfaces between: (i) the bearing shaft 203 and the bearing block 205, and (ii) part of the distal surface of the bearing block 205 and the proximal surface of the retainer ring 206.

TABLE 3
Bearing Designs of the Type Shown in FIG. 3a

| | | | Retainer | Retainer | | | |
|---|---|---|---|---|---|---|---|
| | Drive | Bearing | Ring | Ring | | | |
| | Shaft | Block | Inner | Outer | GF | GF | GF |
| Design | Radius | Length | Radius | Radius | Cylinders | Annulus | Total |
| Name | (mm) | (mm) | (mm) | (mm) | (mm³) | (mm³) | (mm³) |
| Relevant | | | | | Equation | Equation | |
| Equation | | | | | 1 | 2 | |
| F3A001 | 0.100 | 1.00 | 0.10 | 0.70 | 0.03 | 0.72 | 0.75 |
| F3A002 | 0.100 | 2.00 | 0.10 | 0.70 | 0.06 | 0.72 | 0.78 |
| F3A003 | 0.250 | 1.00 | 0.25 | 0.85 | 0.20 | 1.25 | 1.45 |
| F3A004 | 0.250 | 2.00 | 0.25 | 0.85 | 0.39 | 1.25 | 1.65 |
| F3A005 | 0.375 | 1.00 | 0.50 | 1.10 | 0.44 | 2.53 | 2.97 |
| F3A006 | 0.375 | 2.00 | 0.50 | 1.10 | 0.88 | 2.53 | 3.41 |
| F3A007 | 0.500 | 1.00 | 0.75 | 1.35 | 0.79 | 4.27 | 5.05 |
| F3A008 | 0.500 | 2.00 | 0.75 | 1.35 | 1.57 | 4.27 | 5.84 |

It can be seen, the cylindrical bearing surface between the bearing shaft 203 and the bearing block 205 contributes a much smaller part to GF than the annular surface between the bearing block 205 and the retainer ring 206. Design F3A001 has a GF of 0.75 mm³ which is the lowest GF value from either table 2 or table 3. The smaller outer diameter of the bearing surface of the retainer ring 109 has the most influence on the low GF of F3A001.

Table 4 presents the dimensional parameters and GF calculated values for nine designs with the geometric characteristics as illustrated in FIG. 5. The geometric drag factors (GF) for the designs F5001 through to F5009 depend on the bearing surfaces between: (i) the cuff concave bearing 326 and the proximal impeller ball nose 324, and (ii) the distal concave static bearing 327 and the distal impeller ball nose 325. This bearing arrangement 321 has no cylindrical bearing surfaces to maintain the impeller 302 on the central axis of the pump housing 301 in operation. With the design illustrated in FIG. 5a, the proximal impeller ball nose 324, and the distal impeller ball nose 325 are configured to maintain the impeller 302 on the central axis of the pump housing when in operation. This is achieved by ensuring that when the impeller is proximally biased to the extent that the proximal impeller ball nose 324 bears on the cuff concave bearing 326 that the axial gap between the distal concave static bearing 327 and the distal impeller ball nose 325 is less than or equal to 20 micrometers. This limited axial movement has the effect of restraining the impeller 302 on the central axis of the pump housing 301 due to the cupping effect of the cuff concave bearing 326 and the distal concave bearing.

In an embodiment, the angles α1 and α2 are selected such that at least component of the static bearing surfaces restrains the impeller 302 on the central axis of the pump housing. It will be obvious that the bearing surfaces both proximal and distal of the impeller are bearing surfaces, even when the impeller 302 is biased proximally or distally and this is reflected in the calculations in table 4.

TABLE 4
Bearing Designs of the Type Shown in FIG. 5

| | Sphere | First Axial | Second Axial | |
|---|---|---|---|---|
| Design | Radius | Angle (α1) | Angle (α2) | GF |
| Name | (mm) | Deg | Deg | (mm³) |
| Relevant | | | | Equation 5 |
| Equation | | | | |
| F5001 | 0.50 | 8 | 90 | 1.23 |
| F5002 | 0.50 | 8 | 75 | 0.83 |
| F5003 | 0.50 | 8 | 60 | 0.48 |
| F5004 | 0.40 | 10 | 90 | 0.63 |
| F5005 | 0.40 | 10 | 75 | 0.42 |
| F5006 | 0.40 | 10 | 60 | 0.25 |
| F5007 | 0.30 | 15 | 90 | 0.26 |
| F5008 | 0.30 | 15 | 75 | 0.18 |
| F5009 | 0.30 | 15 | 60 | 0.10 |

It will be noted that the GF values in Table 4 are generally lower than those in Table 2 or Table 3 with F5009 achieving a GF of 0.10 mm³. It will further be noted that numbers chosen for each design in Table 3 for (i) the radius of the bearing spherical surfaces, (ii) al and (iii) α2 are the same for the proximal bearing surface and the distal bearing surface. This makes the table simpler. It will be obvious that all of (i) the radius of the bearing spherical surfaces, (ii) α1 and (iii) α2, could be different as between the proximal and distal bearing surfaces.

While preferred embodiments of the present invention are described above, it will be apparent to one skilled in the art that various changes/modifications may be made. The appended claims are intended to cover all such changes and modifications that fall within the true spirit and scope of the invention.

1. An intravascular blood pump assembly comprising:
an elongated tubular housing having a proximal end, a distal end, at least one inlet and at least one outlet;
a pumping element positioned within the elongated tubular housing and configured to rotate relative to the elongated tubular housing; and
a bearing arrangement configured to hold the pumping element and configured geometrically to generate a low bearing interfacial resistive force.

2. The intravascular blood pump assembly of claim 1, wherein the pumping element comprises at least one bearing surface.

3. The intravascular blood pump assembly of claim 2, wherein the bearing surface comprises a geometric drag factor.

4. The intravascular blood pump assembly of claim 2, wherein the bearing surface is configured geometrically to generate a low geometric drag factor.

5. The intravascular blood pump assembly of claim 2, wherein the bearing surface comprises a first bearing surface and a second bearing surface.

6. The intravascular blood pump assembly of claim 5, wherein the first bearing surface and the second bearing surface comprise a continuous surface.

7. The intravascular blood pump assembly of claim 2, wherein the bearing surface comprises a static surface and a rotating surface.

8. The intravascular blood pump assembly of claim 7, wherein the static surface and the rotating surface are substantially equal.

9. The intravascular blood pump assembly of claim 1, wherein the bearing arrangement comprises at least one bearing gap.

10. The intravascular blood pump assembly of claim 9, wherein the bearing gap comprises a small space between the static surface and the rotating surface.

11. The intravascular blood pump assembly of claim 3, wherein the geometric drag factor is less than 2.97 mm⁴.

12. (canceled)

13. (canceled)

14. (canceled)

15. (canceled)

16. The intravascular blood pump assembly of claim 1, wherein the low bearing interfacial resistive force results partially from a low geometric drag factor.

17. The intravascular blood pump assembly of claim 1, wherein the pumping element comprises an impeller and the impeller comprises a pair of radially opposed impeller blades, wherein the impeller blade comprises an impeller blade proximal end, an impeller blade distal end, and a blade angle with respect to an axis of the impeller.

18. (canceled)

19. (canceled)

20. The intravascular blood pump assembly of claim 17, wherein the blade angle of the impeller blade proximal end is configured to accelerate blood radially.

21. (canceled)

22. (canceled)

23. (canceled)

24. (canceled)

25. (canceled)

26. The intravascular blood pump assembly of claim 17 further comprising a bearing shaft positioned distally of the impeller.

27. The intravascular blood pump assembly of claim 1, wherein the bearing arrangement comprises a bearing block.

28. The intravascular blood pump assembly of claim 27, wherein the pumping element comprises an impeller and wherein the bearing block comprises a plurality of bearing surfaces.

29. The intravascular blood pump assembly of claim 28, wherein the bearing block comprises a first distal bearing surface configured to restrain the bearing shaft substantially fixed relative to a neutral axis of the impeller.

30. The intravascular blood pump assembly of claim 29, wherein the bearing block comprises a second distal bearing surface configured to restrain the impeller at least partially in a substantially fixed axial position relative to the bearing block.

31. The intravascular blood pump assembly of claim 30, wherein the bearing block comprises a third distal bearing surface configured to restrain the impeller at least partially in a substantially fixed axial position relative to the bearing block.

32. (canceled)

33. (canceled)

34. (canceled)

35. (canceled)

36. (canceled)

37. (canceled)

38. The intravascular blood pump assembly of claim 28, wherein the bearing arrangement comprises a proximal bearing surface proximal of the impeller.

39. The intravascular blood pump assembly of claim 38, further comprising a motor and a drive shaft extending between the motor and the impeller, wherein the proximal bearing surface is configured to restrain the drive shaft substantially fixed relative to the neutral axis of the impeller.

40. The intravascular blood pump assembly of claim 38, wherein the proximal bearing surface comprises a plurality of bearing surface segments disposed equiangularly around the drive shaft.

41. (canceled)

42. (canceled)

43. (canceled)

44. (canceled)

45. An intravascular blood pump assembly comprising:
an elongate tubular housing having a proximal end, a distal end, at least one inlet, and at least one outlet;
an impeller positioned within the elongate tubular housing and configured to rotate relative to the elongate tubular housing; and
a bearing block distal of the impeller having a first bearing surface and a second bearing surface,
wherein the first bearing surface defines an axis of restraint, and the second bearing surface defines a plane of restraint, wherein the axis of restraint and the plane of restraint are orthogonal to each other.

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. An intravascular catheter blood pump comprising:
a pump assembly, a catheter shaft, a drive shaft, and a motor,
wherein the pump assembly comprises a pump housing, an impeller, a restrictor, a pump inlet, and a pump outlet downstream of the pump inlet and the restrictor,
wherein the drive shaft is coaxially connected to the impeller at its distal end and coaxially coupled to a rotor shaft of the motor at its proximal end and configured to transmit torque or rotational energy from the motor to the impeller,
wherein the catheter shaft comprises a central lumen configured to receive the drive shaft and facilitate the rotational operation of the drive shaft.

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. (canceled)

85. (canceled)

86. (canceled)

87. (canceled)

88. (canceled)

89. (canceled)

90. (canceled)

91. (canceled)

92. (canceled)

93. (canceled)
